(19) United States
(12) Patent Application Publication
(10) Pub. No.: US 2026/0075310 A1
(43) Pub. Date: Mar. 12, 2026

(54) MEDICAL IMAGE CAPTURING SYSTEM AND IMAGING METHOD

(71) Applicant: Sony Olympus Medical Solutions Inc., Tokyo (JP)

(72) Inventor: Masashi MURAKITA, Tokyo (JP)

(73) Assignee: Sony Olympus Medical Solutions Inc., Tokyo (JP)

(21) Appl. No.: 19/312,364

(22) Filed: Aug. 28, 2025

(30) Foreign Application Priority Data
Sep. 10, 2024 (JP) 2024-156858
Mar. 6, 2025 (JP) 2025-035796

(51) Int. Cl. H04N 23/60; A61B 1/00; A61B 1/045; A61B 1/06; H04N 23/74

(52) U.S. Cl. CPC H04N 23/64; A61B 1/00009; A61B 1/00096; A61B 1/045; A61B 1/0655; H04N 23/74

(57) ABSTRACT

A medical image capturing system captures imaging light including a subject image obtained by irradiating a body cavity with illumination light from a light source unit, and includes: an imaging unit that captures, via a scope, a captured image including an imaging region where the imaging light reaches an imaging surface and a mask region where the imaging light does not reach the imaging surface; an exposure state determination unit that determines an exposure state on the basis of information of pixels within a predetermined range of the captured image; and a processing unit that executes processing by setting a type of the mask region as a first mask type determined on the basis of the captured image or a second mask type determined on the basis of an image acquired before the imaging according to the exposure state.

### CROSS-REFERENCE TO RELATED APPLICATIONS

This application claims the benefit of Japanese Priority Patent Application JP 2024-156858 filed on Sep. 10, 2024, and Japanese Priority Patent Application JP 2025-035796 filed on Mar. 6, 2025, the entire contents of each are incorporated herein by reference.

### TECHNICAL FIELD

The present disclosure relates to a medical image capturing system and an imaging method.

### BACKGROUND ART

In general, a surgical endoscope uses an imaging device to which a scope is connected, and observes an operative field by inserting the scope into a patient. The scope is detachable, and a scope to be used is selected from a plurality of types of scopes. At this time, depending on the type of the scope, an imaging region in which the imaging light reaches the imaging surface of the imaging device is different from a mask region in which the imaging light does not reach the imaging surface of the imaging device. Furthermore, in a processing unit using the image information in the imaging region, it is necessary to adjust the acquisition range of the image information according to the range of the imaging region in order to increase the processing accuracy. Therefore, a method for determining information corresponding to a mask type of the mask region is known (see, for example, JP 2004-33487 A).

### CITATION LIST

### Patent Literature

[PTL 1]

JP 2004-33487 A

### SUMMARY

### Technical Problem

However, in a case where the distal end portion of the scope is close to the imaging target, or the like, imaging light incident on the scope from the imaging target rapidly increases, and may reach the imaging surface of the mask region beyond the boundary between the imaging region and the mask region. In such a case, there is a possibility that the mask type is erroneously determined. Therefore, the processing unit that executes the processing based on the mask type may decrease the processing accuracy.

The present disclosure provides a medical image capturing system and a medical image capturing method capable of suppressing a decrease in processing accuracy of a processing unit that executes processing based on a mask type even in a case where imaging light incident on a scope from an imaging target rapidly increases.

### Solution to Problem

In order to solve the above problem, according to the present disclosure, there is provided a medical image capturing system that captures imaging light including a subject image obtained by irradiating a body cavity with illumination light from a light source unit. The medical image capturing system includes: an imaging unit that captures, via a scope, a captured image including an imaging region where the imaging light reaches an imaging surface and a mask region where the imaging light does not reach the imaging surface; an exposure state determination unit that determines an exposure state on the basis of information of pixels within a predetermined range of the captured image; and a processing unit that executes processing by setting a type of the mask region as a first mask type determined on the basis of the captured image or a second mask type determined on the basis of an image acquired before the imaging according to the exposure state.

The exposure state determination unit may determine whether or not the type of the mask region can be correctly recognized.

In a case where the exposure state determination unit determines that the exposure state is a state in which the type of the mask region can be correctly recognized, the processing unit may perform processing based on the first mask type.

In a case where the exposure state determination unit determines that the exposure state is a state in which the type of the mask region cannot be correctly recognized, the processing unit may perform processing based on the second mask type.

The processing unit may control a light emission amount of the light source unit according to a detection region corresponding to a type of the mask region.

The processing unit may reduce the light emission amount by PWM control.

The imaging unit may capture the captured image via a focus lens, and the processing unit may control a focus of the focus lens according to a type of the mask region.

The processing unit may stop focus control of the focus lens in a case where the exposure state determination unit determines that the exposure state is a state in which a type of the mask region cannot be correctly recognized.

The processing unit may control a gain of a signal output from the imaging unit according to a type of the mask region.

The processing unit may execute gradation conversion of the captured image according to a type of the mask region.

In a case where the exposure state determination unit determines that the exposed state is a state in which a type of the mask region cannot be correctly recognized, the processing unit may execute gradation conversion for widening a dynamic range more than that at a time of the determination.

The exposure state determination unit may determine that the exposure state is a state in which a type of the mask region cannot be correctly recognized at least in a case where the first mask type and the second mask type are different and an evaluation value based on a pixel value in an evaluation frame in a central portion of the captured image is larger than a predetermined value.

A setting unit may be further included which sets a plurality of evaluation frames such that a mask boundary between the mask region of the first mask type and the imaging region is between the plurality of evaluation frames.

The exposure state determination unit may set, as a condition that the exposure state is a state in which a type of the mask region cannot be correctly recognized, that an evaluation value in the evaluation frame on the imaging region side of the two evaluation frames sandwiching the mask boundary of the first mask type is a predetermined value or more under control of the processing unit.

The exposure state determination unit may set, as a condition that the exposure state is a state in which a type of the mask region cannot be correctly recognized, that a pixel value average in an evaluation frame in a central portion of the captured image is a predetermined value or more.

The exposure state determination unit may set, as a condition that the exposure state is a state in which a type of the mask region can be correctly recognized, that an evaluation value in the evaluation frame on the mask region side of the two evaluation frames sandwiching the mask boundary of the first mask type is a predetermined value or less under control of the processing unit.

The setting unit may set the plurality of evaluation frames to be point-symmetric with respect to a corresponding point of an optical axis of the scope.

An estimation unit may be further included which estimates a type of the scope on the basis of a type of the mask region.

In order to solve the above problem, according to the present disclosure, there is provided a method of capturing a medical image that captures imaging light including a subject image obtained by irradiating a body cavity with illumination light from a light source unit.

The method includes: capturing, via a scope, a captured image including an imaging region where the imaging light reaches an imaging surface and a mask region where the imaging light does not reach the imaging surface; determining an exposure state on the basis of information of pixels within a predetermined range of the captured image; and executing processing by setting a type of the mask region as a first mask type determined on the basis of the captured image or a second mask type determined on the basis of an image acquired before the imaging according to the exposure state.

### BRIEF DESCRIPTION OF DRAWINGS

FIG. 1 is a diagram illustrating an example of a schematic configuration of a medical image capturing system to which a technology according to the present disclosure is applied.

FIG. 2 is a diagram illustrating an example of a detailed configuration of a medical image capturing device of FIG. 1.

FIG. 3 is a block diagram illustrating a configuration example of a camera head, a CCU, and a light source device illustrated in FIGS. 1 and 2.

FIG. 4 is a diagram illustrating an example of a captured image (endoscopic image) according to an image signal.

FIG. 5 is a diagram illustrating a treatment example of an operator.

FIG. 6 is a block diagram illustrating a configuration example of a control unit.

FIG. 7 is a block diagram illustrating a configuration example of a photometry unit.

FIG. 8 is a diagram illustrating an example of an evaluation frame for determining whether a mask region is included.

FIG. 9 is a diagram illustrating an example of an evaluation frame for determining the type of mask region.

FIG. 10 is a diagram illustrating an example of a light amount detection region for light amount control.

FIG. 11 is a block diagram illustrating a configuration example of an imaging control unit.

FIG. 12 is a diagram illustrating an example of light emission adjustment from a case where a target light amount of a light source device is maximum to a case where the target light amount of the light source device is minimum.

FIG. 13 is a diagram illustrating a relationship between an analog gain of an imaging unit and an evaluation value generated by a light reception value generation unit.

FIG. 14 is a block diagram illustrating a configuration example of a determination processing unit.

FIG. 15 is a flowchart of a first processing example.

FIG. 16 is a flowchart of a second processing example. FIG. 17

FIGS. 17A and 17B are diagrams illustrating a mask type ty4 and a mask type ty3.

FIG. 18 is a diagram illustrating an example of an evaluation frame of the mask type ty4.

FIG. 19 is a diagram illustrating an example of an evaluation frame of the mask type ty3.

FIG. 20 is a state transition diagram illustrating a determination result of a mask type.

FIG. 21 is a diagram illustrating an example of an evaluation frame of the mask type ty3.

FIG. 22 is a diagram illustrating an example of an evaluation frame of the mask type ty2.

FIG. 23 is a time chart illustrating a transition of a first mode from an OFF state to an ON state. FIG. 24

FIGS. 24A and 24B are diagrams illustrating an example of a captured image in a first state.

FIG. 25 is a flowchart illustrating a process example after the determination processing of FIG. 15.

### DESCRIPTION OF EMBODIMENTS

Hereinafter, embodiments of the present disclosure will be described with reference to the drawings.

### First Embodiment

### (Outline of System)

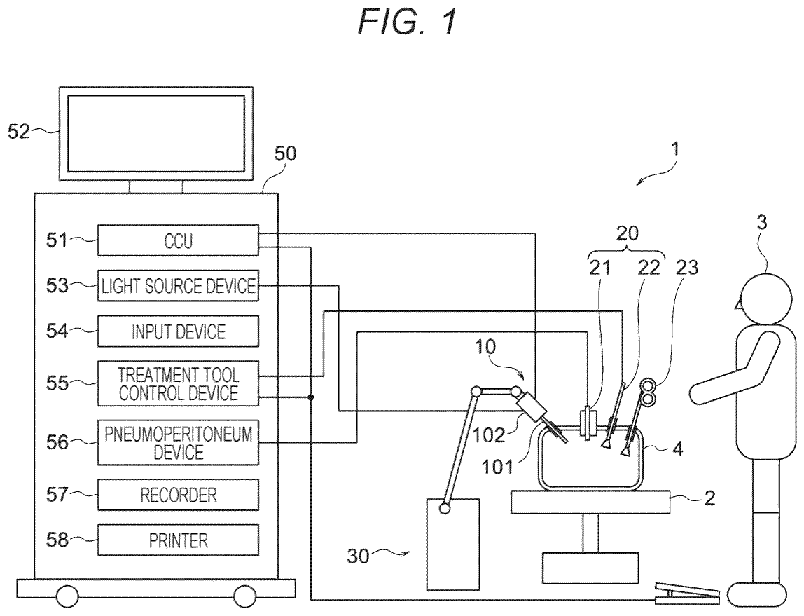

First, an outline of a system to which the technology according to the present disclosure can be applied will be described. FIG. 1 illustrates an example of a schematic configuration of a medical image capturing system to which the technology according to the present disclosure is applied.

FIG. 1 illustrates a state in which an operator (doctor) 3 is performing surgery on a patient 4 on a patient bed 2 using a medical image capturing system 1. As illustrated in FIG. 1, a medical image capturing system 1 is a system that captures imaging light including a subject image obtained by irradiating an inside of a subject (inside of a patient) with illumination light from a light source unit.

The medical image capturing system 1 includes a medical image capturing device 10, other surgical tools 20 such as a pneumoperitoneum tube 21, an energy treatment tool 22, and forceps 23, a support arm device 30 that supports the medical image capturing device 10, a treatment tool control device 55 in a cart 50 on which various devices for endoscopic surgery are mounted, a pneumoperitoneum device 56, a recorder 57, and a printer 58. The medical image capturing device 10 includes, for example, a camera control unit (CCU) 51, a display device 52, a light source device 53, and an input device 54 in the cart 50, a scope 101, and a camera head 102.

A region of a predetermined length from the distal end of a scope (endoscope) 101 is inserted into a body cavity of the patient 4. The camera head 102 is connected to the proximal end of the scope 101. A plurality of types of scopes 101 can be connected to the camera head 102. Note that FIG. 1 illustrates the medical image capturing device 10 configured as a so-called rigid scope including the scope 101 as a rigid lens barrel, but the medical image capturing device 10 may be configured as a so-called flexible scope including a flexible lens barrel.

An opening into which an objective lens is fitted is provided at the distal end of the scope 101. The light source device 53 is connected to the medical image capturing device 10, and light (irradiation light) generated by the light source device 53 is guided to the distal end of the lens barrel by a light guide extending inside the scope 101, and is emitted toward an observation target in the body cavity of the patient 4 via the objective lens. Note that the medical image capturing device 10 may be a forward-viewing endoscope, an oblique-viewing endoscope, or a side-viewing endoscope.

An optical system and an imaging element are provided inside the camera head 102, and imaging light (observation light) including a subject image reflected from an observation target is condensed on an imaging surface of the imaging element in the imaging unit by the optical system. In this manner, the imaging light including the subject image reaches the imaging surface via any of the plurality of scopes 101. Furthermore, an imaging region where imaging light reaches the imaging surface and a mask region where imaging light does not reach the imaging surface are generated by the optical path shape of the scope 101.

The imaging element photoelectrically converts the observation light to generate an image signal corresponding to the subject image. This image signal is transmitted to a camera control unit (CCU) 51 as RAW data (captured image). As described above, the imaging unit of the camera head 102 captures the captured image including the imaging region where the imaging light reaches the imaging surface and the mask region where the imaging light does not reach the imaging surface via any one of the plurality of scopes 101.

The CCU 51 includes a processor such as a central processing unit (CPU) or a graphics processing unit (GPU), for example, and integrally controls the operations of the medical image capturing device 10 and the display device 52. Moreover, the CCU 51 receives an image signal from the camera head 102, and performs various types of image processing for displaying an observation image (display image) based on the image signal, such as gradation conversion processing using a gamma curve (gradation conversion processing curve) and development processing (demosaic processing), on the image signal.

The display device 52 displays a display image based on the image signal subjected to the image processing by the CCU 51 under the control of the CCU 51.

The light source device 53 includes a light source such as a light emitting diode (LED), for example, and supplies irradiation light for photographing a surgical site or the like to the medical image capturing device 10.

The input device 54 is an input interface for the medical image capturing system 1. The user can input various types of information and instructions to the medical image capturing system 1 via the input device 54. For example, the user inputs an instruction or the like to change imaging conditions (type, magnification, focal length, and the like of irradiation light) by the medical image capturing device 10.

The treatment tool control device 55 controls driving of the energy treatment tool 22 for cauterization and incision of tissue, sealing of a blood vessel, or the like. The pneumoperitoneum device 56 sends gas into the body cavity via the pneumoperitoneum tube 21 in order to inflate the body cavity of the patient 4 for the purpose of securing a visual field by the medical image capturing device 10 and securing a working space of the operator.

The recorder 57 is a device capable of recording various types of information regarding surgery. The printer 58 is a device capable of printing various types of information regarding surgery in various formats such as text, image, or graph.

### (Configuration of Medical Image Capturing Device)

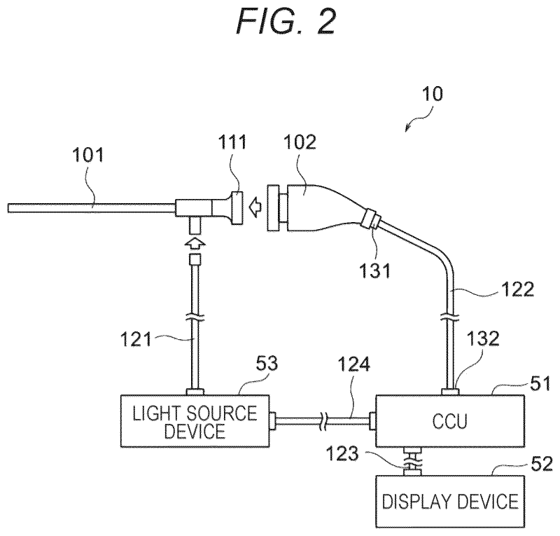

FIG. 2 illustrates an example of a detailed configuration of the medical image capturing device 10 of FIG. 1.

Here, the CCU 51, the display device 52, the light source device 53, the scope 101, and the camera head 102 are illustrated.

As illustrated in FIG. 2, in the medical image capturing device 10, the scope 101 is connected to the light source device 53 via a light guide 121, and the camera head 102 is connected to the CCU 51 via a transmission cable 122. Moreover, the CCU 51 is connected to the display device 52 via a transmission cable 123 and is connected to the light source device 53 via a transmission cable 124.

The scope 101 is configured as a rigid scope. That is, the scope 101 is an insertion portion (lens barrel) that is rigid or at least partially soft and has an elongated shape, and is inserted into the body cavity of the patient 4. In the scope 101, an optical system that is configured using one or a plurality of lenses and collects a subject image is provided.

As described above, the light source device 53 is connected to one end of the light guide 121, and supplies the irradiation light for illuminating the body cavity to one end of the light guide 121 under the control of the CCU 51. One end of the light guide 121 is detachably connected to the light source device 53, and the other end is detachably connected to the scope 101.

Then, the light guide 121 transmits the irradiation light supplied from the light source device 53 from one end to the other end, and supplies the irradiation light to the scope 101. As described above, the irradiation light supplied to the scope 101 is emitted from the distal end of the scope 101 and is applied to the body cavity.

Observation light (subject image) emitted into the body cavity and reflected in the body cavity is collected by the optical system in the scope 101.

As described above, the camera head 102 is detachably connected to the proximal end (eyepiece unit 111) of the scope 101. Then, under the control of the CCU 51, the camera head 102 captures the imaging light (subject image) condensed by the scope 101, and outputs an image signal (captured image data) obtained as a result. The image signal is, for example, an image signal corresponding to 4K resolution (for example, 3840×2160 pixels). Note that a detailed configuration of the camera head 102 will be described later. Furthermore, in the present embodiment, the captured image data may be referred to as a captured image.

One end of the transmission cable 122 is detachably connected to the camera head 102 via a connector 131, and the other end is detachably connected to the CCU 51 via a connector 132. Then, the transmission cable 122 transmits the image signal and the like output from the camera head 102 to the CCU 51, and transmits a control signal, a synchronization signal, power, and the like output from the CCU 51 to the camera head 102.

Note that, for transmission of an image signal or the like from the camera head 102 to the CCU 51 via the transmission cable 122, an image signal or the like may be transmitted as an optical signal or may be transmitted as an electric signal. The same applies to transmission of a control signal, a synchronization signal, and a clock from the CCU 51 to the camera head 102 via the transmission cable 122. Furthermore, communication between the camera head 102 and the CCU 51 is not limited to wired communication using the transmission cable 122, and wireless communication conforming to a predetermined communication method may be performed.

Under the control of the CCU 51, the display device 52 displays a display image based on the image signal from the CCU 51, and outputs sound according to a control signal from the CCU 51.

One end of the transmission cable 123 is detachably connected to the display device 52, and the other end is detachably connected to the CCU 51. Then, the transmission cable 123 transmits the image signal processed by the CCU 51 and the control signal output from the CCU 51 to the display device 52.

The CCU 51 includes a CPU and the like, and integrally controls operations of the light source device 53, the camera head 102, and the display device 52. Note that a detailed configuration of the CCU 51 will also be described later.

One end of the transmission cable 124 is detachably connected to the light source device 53, and the other end is detachably connected to the CCU 51. Then, the transmission cable 124 transmits the control signal from the CCU 51 to the light source device 53.

### (Configurations of Camera Head, CCU, and Light Source Device)

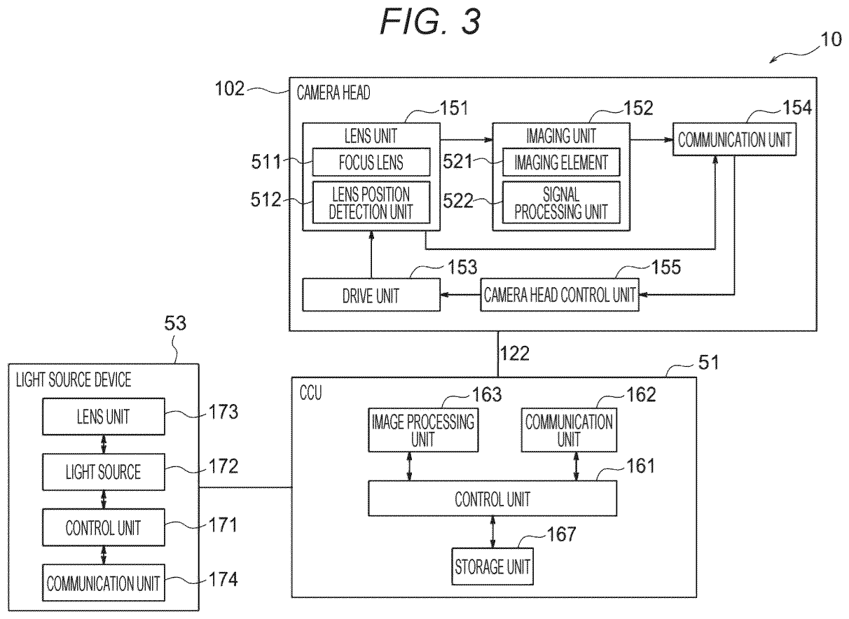

FIG. 3 is a block diagram illustrating a configuration example of the camera head 102, the CCU 51, and the light source device 53 illustrated in FIGS. 1 and 2.

The camera head 102 includes a lens unit 151, an imaging unit 152, a drive unit 153, a communication unit 154, and a camera head control unit 155. The camera head 102 and the CCU 51 are communicably connected to each other by the transmission cable 122.

The lens unit 151 is configured using a plurality of lenses movable along the optical axis, and forms a subject image condensed by the scope 101 on an imaging surface of an imaging element 521 (see FIG. 3) of the imaging unit 152. The lens unit 151 includes a focus lens 511 and a lens position detection unit 512. The focus lens 511 includes one or a plurality of lenses, and adjusts the focus by moving along the optical axis.

Furthermore, the lens unit 151 is provided with a focus mechanism (not illustrated) that moves the focus lens 511 along the optical axis.

The lens position detection unit 512 is configured using a position sensor such as a photo interrupter, and detects a lens position (hereinafter, referred to as a focus position) of the focus lens 511. Then, the lens position detection unit 512 outputs a detection signal corresponding to the focus position to the CCU 51 via the communication unit 154.

The imaging unit 152 includes an imaging element 521 and a signal processing unit 522. The imaging element 521 includes a charge coupled device (CCD), a complementary metal oxide semiconductor (CMOS), or the like that receives a subject image collected by the scope 101 and formed by the lens unit 151 and converts the subject image into an electric signal (analog signal).

The number of imaging elements 521 may be one (so-called single plate type) or plural (so-called multi-plate type). In a case where the imaging unit 152 is configured as a multi-plate type, for example, image signals corresponding to RGB may be generated by the respective imaging elements, and a color image may be obtained by combining the image signals.

Alternatively, the imaging unit 152 may include a pair of imaging elements 521 for acquiring right-eye and left-eye image signals corresponding to three-dimensional (3D) display. By performing the 3D display, the operator 3 can more accurately grasp the depth of the living tissue in the surgical site. Note that, in a case where the imaging unit 152 is configured as a multi-plate type, a plurality of lens units 151 can be provided corresponding to the respective imaging elements 521.

The signal processing unit 522 performs signal processing on the electric signal (analog signal) from the imaging element 521 and outputs an image signal. For example, the signal processing unit 522 performs, on the electric signal (analog signal) from the imaging element 521, signal processing such as processing of removing reset noise, processing of multiplying an analog gain for amplifying the analog signal, and A/D conversion.

The drive unit 153 includes an actuator or the like, and moves a zoom lens or a focus lens included in the lens unit 151 by a predetermined distance along the optical axis under the control of the camera head control unit 155. Therefore, the magnification and focus of the image captured by the imaging unit 152 can be appropriately adjusted.

The communication unit 154 includes a communication device for transmitting and receiving various types of information to and from the CCU 51. The communication unit 154 transmits the image signal obtained from the imaging unit 152 as RAW data to the CCU 51 via the transmission cable 122.

Furthermore, the communication unit 154 receives a control signal for controlling driving of the camera head 102 from the CCU 51, and supplies the control signal to the camera head control unit 155. The control signal includes, for example, information regarding imaging conditions such as information for specifying a frame rate of an image, information for specifying an exposure value at the time of imaging, or information for specifying a magnification and a focus of an image.

The light source device 53 includes a control unit 171, a light source 172, a lens unit 173, and a communication unit 174. The control unit 171 of the light source device 53 controls the light source 172 and the lens unit 173 under the control of the CCU 51. In the present example, the light emitted by the light source 172 travels through the lens unit 173 towards the light guide 121. The light emission amount from the light source 172 can be adjusted by, for example, pulse modulation control of a current drive pulse. That is, the light emission amount of the light source device 53 can be adjusted by PWM control by the control unit 171.

Note that the imaging conditions such as the frame rate, the exposure value, the magnification, and the focus may be appropriately designated by the user, or may be automatically set by a control unit 161 of the CCU 51 on the basis of the acquired image signal. That is, in the latter case, the medical image capturing device 10 has a so-called auto exposure (AE) function, an auto focus (AF) function, and an auto white balance (AWB) function.

The camera head control unit 155 controls driving of the camera head 102 on the basis of the control signal from the CCU 51 received via the communication unit 154.

The CCU 51 is an image processing device including a control unit 161, a communication unit 162, an image processing unit 163, and a storage unit 167. The control unit 161 performs various types of control related to imaging of a surgical site or the like by the medical image capturing device 10 and displaying of an endoscopic image (medical captured image) obtained by imaging of the surgical site or the like. For example, the control unit 161 generates a control signal for controlling driving of the camera head 102. Similarly, the control unit 161 generates a control signal for controlling the light amount of the light source 172.

Furthermore, the control unit 161 causes the display device 52 to display a display image (endoscopic image) of a surgical site or the like on the basis of the image signal subjected to the image processing by the image processing unit 163. At this time, the control unit 161 can cause the image processing unit 163 to change the curve shape of the gamma curve (gradation conversion curve) of the endoscopic image to generate the display image. Furthermore, the control unit 161 may recognize various objects in the image using various image recognition technologies.

For example, the control unit 161 can recognize a surgical tool such as forceps, a specific body part, bleeding, mist at the time of using the energy treatment tool 22, and the like by detecting the shape, color, and the like of the edge of the object included in the image.

When displaying the image on the display device 52, the control unit 161 may superimpose and display various types of surgery support information on the image of the surgical site using the recognition result. Since the surgery support information is superimposed and displayed and presented to the operator 3, the burden on the operator 3 can be reduced and the operator 3 can reliably proceed with the surgery. Furthermore, the control unit 161 detects proximity saturation on the basis of the image captured by the imaging unit 152.

The communication unit 162 includes a communication device for transmitting and receiving various types of information to and from the camera head 102. The communication unit 162 receives an image signal transmitted from the camera head 102 via the transmission cable 122.

Furthermore, the communication unit 162 transmits a control signal for controlling driving of the camera head 102 to the camera head 102. The image signal and the control signal can be transmitted by electric communication, optical communication, or the like.

The image processing unit 163 performs various types of image processing on the image signal including the RAW data transmitted from the camera head 102. The storage unit 167 stores various control parameters and the like used by the control unit 161.

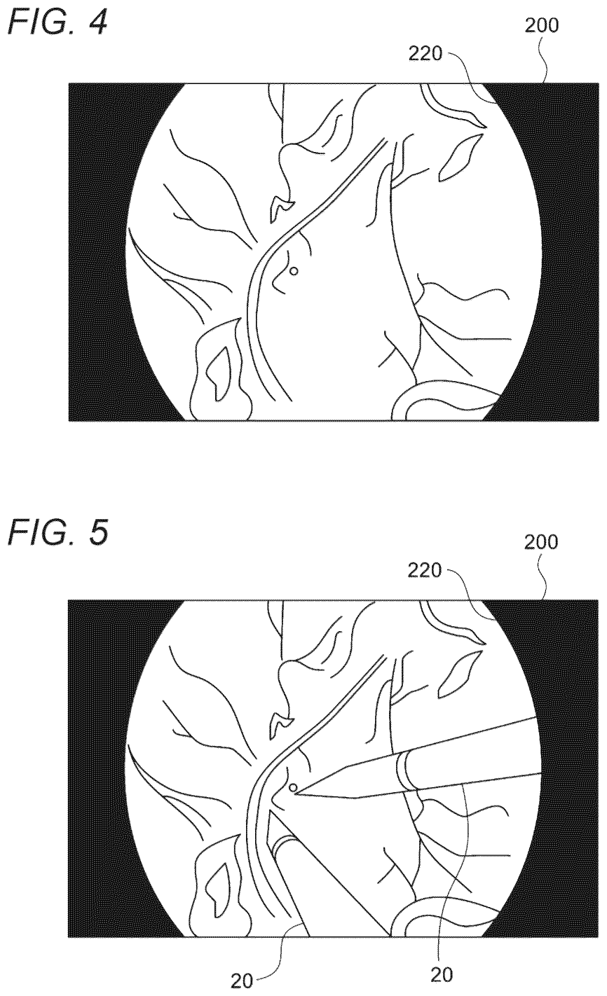

Meanwhile, as described above, in the medical image capturing device 10, the scope 101 connected to the camera head 102 is inserted into the body cavity of the patient 4, whereby the operator 3 observes the operative field. For example, FIG. 4 illustrates an example of a captured image (endoscopic image) 200 according to an image signal obtained by imaging the subject image collected by the scope 101 using the camera head 102.

Note that, in the present embodiment, the captured image 200 may be referred to as a medical captured image.

In the medical image capturing device 10, the scope 101 having an elongated shape is attached. However, since the shape of the imaging surface of the imaging element of (the imaging unit 152 of) the camera head 102 does not match the shape of the subject image collected by the scope 101, mechanical vignetting occurs due to the scope 101. Therefore, in the captured image 200, left and right black regions represent mask regions (black regions) where mechanical vignetting occurs. In this manner, an imaging region where the imaging light reaches the imaging surface and a mask region where the imaging light does not reach the imaging surface are generated.

A boundary between the imaging region and the mask region (black region) is defined as a mask boundary 220.

Furthermore, the range of the imaging region where the imaging light reaches the imaging surface and the range of the mask region where the imaging light does not reach the imaging surface are different depending on the type of the scope 101. Note that the mask boundary 220 may be referred to as an edge.

Note that the captured image 200 is displayed by the display device 52 as a display image by being subjected to various types of image processing. FIG. 5 is a diagram illustrating a treatment example of an operator.

For example, as illustrated in FIG. 5, the operator 3 can perform treatment such as resection of an affected part using the surgical tool 20 such as the energy treatment tool 22 while viewing the display image in real time.

Here, in the medical image capturing device 10, the scope 101 is detachable, and the scope 101 to be used is selected from a plurality of types of scopes. At this time, mechanical vignetting and properties are different depending on the type of the scope 101 to be used, and the range of the mask region is different as described above. Each of the plurality of scopes 101 has a different mask region. In the present embodiment, each of a plurality of different mask regions may be referred to as a mask type or a type. As will be described later, a type ty1, a type ty2, a type ty3, and a type ty4 are set in descending order of the diameter of the scope 101.

The range of the mask region varies depending on the diameter of the scope 101. That is, the range of the imaging region excluding the mask region from the captured image 200 varies depending on the diameter of the scope 101. For example, in the present embodiment, an example of four types of scopes 101 having different diameters will be described. Note that the number of scopes 101 is an example and is not limited to four types.

For this reason, since it is necessary to adjust image processing in the subsequent stage depending on the type of the scope 101, a method for determining the type of the scope 101 to be used is required. For example, signal processing related to light amount control (AE), focus control (AF), and the like is performed on an imaging region that is a region of a subject image, and various problems occur when focusing or exposure is performed including a mask region. Therefore, it is necessary to determine the type of mask type. Note that, since there is a correspondence relationship between the mask type and the type of the scope 101, it is also possible to determine the type of the scope 101 when the mask type is determined. Hereinafter, details of the present technology will be described with reference to the drawings.

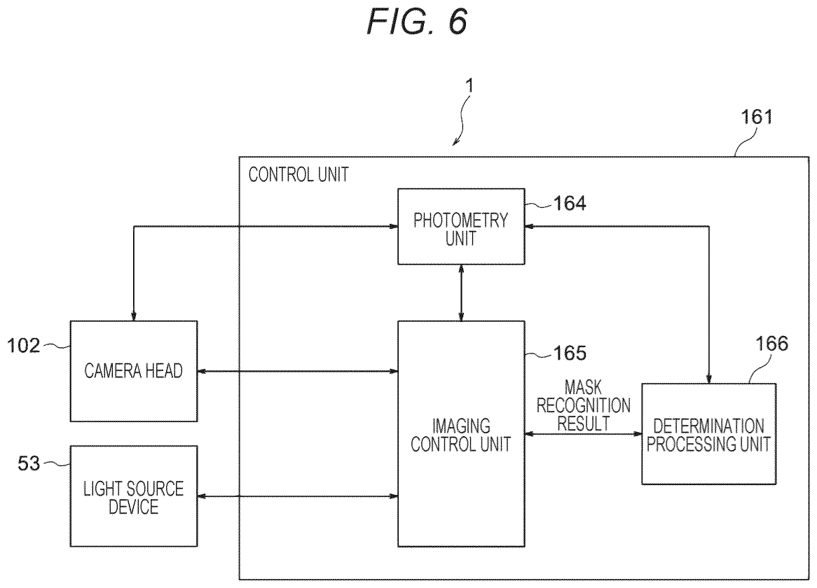

FIG. 6 is a block diagram illustrating a configuration example of the control unit 161. The control unit 161 includes a photometry unit 164, an imaging control unit 165, and a determination processing unit 166. The photometry unit 164 sets a plurality of evaluation frames for determining a mask type, a light amount detection region for light amount control (AE), and a focus detection region for focus control (AF). Furthermore, the photometry unit 164 calculates an evaluation value based on the value of the captured image in each evaluation frame.

The determination processing unit 166 determines the mask type on the basis of the evaluation value generated by the photometry unit 164. The imaging control unit 165 controls the camera head 102 and the light source device 53 on the basis of the evaluation value generated by the photometry unit 164. Note that details of the imaging control unit 165 and the determination processing unit 166 will also be described later. Note that, in the present embodiment, the evaluation value may be referred to as a detection value. Furthermore, the evaluation frame may be referred to as a detection frame, and the evaluation region may be referred to as a detection region.

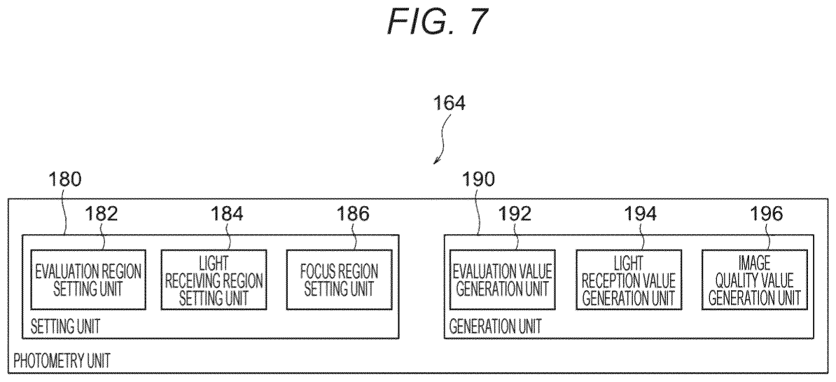

Here, the photometry unit 164 will be described in detail with reference to FIGS. 7 to 10. FIG. 7 is a block diagram illustrating a configuration example of the photometry unit 164. As illustrated in FIG. 7, the photometry unit 164 includes a setting unit 180 and a generation unit 190. As described above, the setting unit 180 sets a plurality of evaluation frames for determining the mask type, a plurality of light amount detection regions for evaluation values, and a focus detection region. That is, the setting unit 180 includes an evaluation region setting unit 182, a light receiving region setting unit 184, and a focus region setting unit 186.

The generation unit 190 generates evaluation values corresponding to the pixel values in the plurality of evaluation frames. That is, the generation unit 190 includes an evaluation value generation unit 192, a light reception value generation unit 194, and an image quality value generation unit 196. The generation unit 190 executes processing according to the mask type and the state determined by the determination processing unit 166.

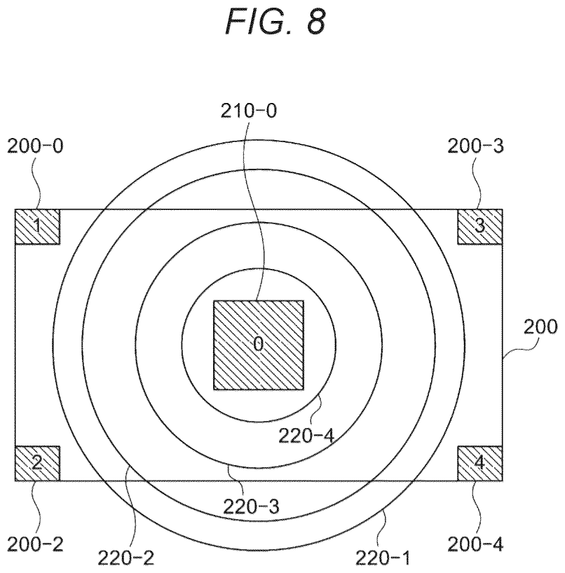

FIG. 8 is a diagram illustrating an example of an evaluation frame for determining whether a mask region is included. In a case where it is determined whether a mask region is included, the evaluation region setting unit 182 sets an evaluation frame 210-0 and evaluation frames 200-0 to 200-4 at four corners of the central portion and the peripheral portion of the captured image 200. As described above, the type ty1, the type ty2, the type ty3, and the type ty4 are set in descending order of the diameter of the scope 101. In FIG. 8, mask boundaries of the type ty1, the type ty2, the type ty3, and the type ty4 are illustrated by circular lines 220-0 to 4. In the following description, in order to simplify the description, the mask boundary may be indicated by a numerical value coming next to a hyphen. Similarly, the evaluation frame, the light amount detection region, and the focus detection region may be indicated by numerical values coming next to a hyphen.

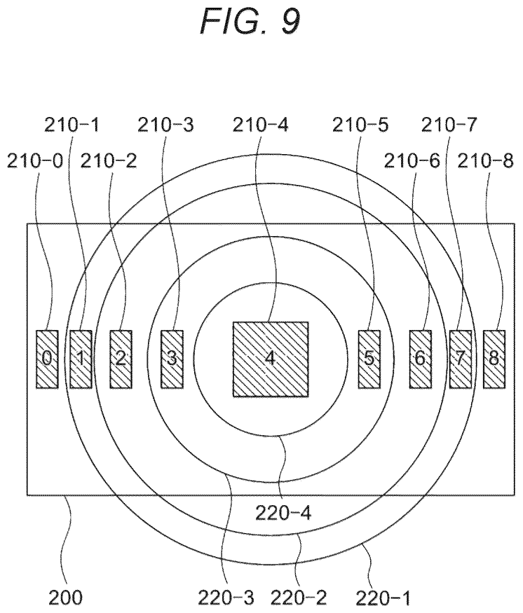

FIG. 9 is a diagram illustrating an example of an evaluation frame for determining the type of mask region. In the evaluation region setting unit 182, rectangular evaluation frames 210-0 to 210-3 (hereinafter, the evaluation frames are also abbreviated as evaluation frames 0, 1, 2, and 3) are provided at predetermined intervals on the left side in the horizontal direction, and rectangular evaluation frames 210-5 to 210-8 (hereinafter, the evaluation frames are also abbreviated as evaluation frames 5, 6, 7, and 8) are provided at predetermined intervals on the right side in the horizontal direction so as to be substantially symmetric about the optical axis center of the scope 101 or the center of gravity of the captured image. The sizes of the rectangles of the evaluation frames 210-0 to 210-3 and the evaluation frames 210-5 to 210-8 discretely arranged at predetermined intervals on the left and right of the central portion are smaller than the size of the rectangle of the evaluation frame 210-4 arranged in the central portion. The mask boundary for each mask region is known in design. Therefore, the evaluation region setting unit 182 sets the plurality of evaluation frames 210-0 to 210-4 such that the mask boundaries 220-0 to 4 of the mask regions of types ty1 to ty4 are between the plurality of evaluation frames 210-0 to 210-4.

Similarly, the evaluation region setting unit 182 sets the plurality of evaluation frames 210-4 to 210-8 such that the mask boundaries 220-0 to 4 of the mask regions of types ty1 to ty4 are between the plurality of evaluation frames 210-4 to 210-8.

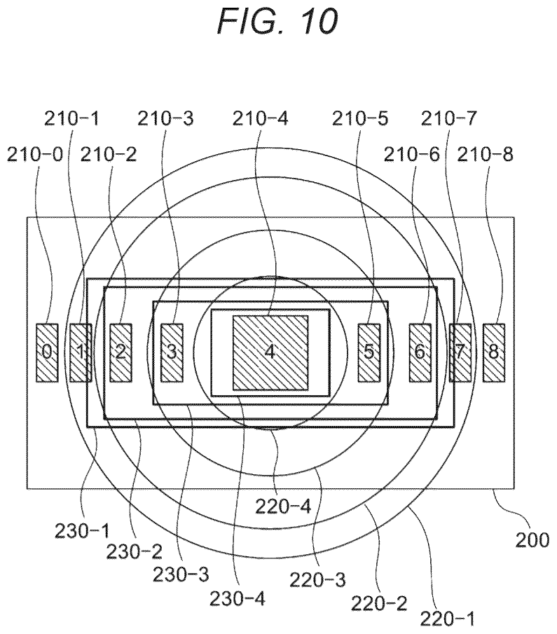

FIG. 10 is a diagram illustrating an example of a light amount detection region for light amount control. The light receiving region setting unit 184 sets the light amount detection regions 230-1 to 4 in association with the mask types ty1 to ty4. The light amount detection regions 230-1 to 4 are set to include at least a part of the evaluation frames in the captured image 200 among the two evaluation frames sandwiching the mask boundary corresponding to the mask types ty1 to ty4.

For example, the light amount detection region 230-4 is set to include the evaluation frame 210-4 in the imaging region of the captured image 200 among the two evaluation frames 210-3 and 210-4 and the two evaluation frames 210-4 and 210-5 sandwiching the mask boundary 220-4 corresponding to the mask type ty4. Similarly, the light amount detection region 230-3 is set to include the evaluation frame 210-3 and 210-5 in the imaging region of the captured image 200 among the two evaluation frames 210-2 and 210-3 and the two evaluation frames 210-5 and 210-6 sandwiching the mask boundary 220-3 corresponding to the mask type ty3. Similarly, the light amount detection region 230-2 is set to include the evaluation frames 210-2 and 210-6 in the imaging region of the captured image 200 among the two evaluation frames 210-1 and 210-2 and the two evaluation frames 210-6 and 210-7 sandwiching the mask boundary 220-1 corresponding to the mask type ty2. Similarly, the light amount detection region 230-1 is set so as to include at least a part of the evaluation frames 210-1 and 210-7 in the imaging region of the captured image 200 among the two evaluation frames 210-0 and 210-1 and the two evaluation frames 210-7 and 210-8 sandwiching the mask boundary 220-1 corresponding to the mask type ty1. In this manner, the light amount detection regions 230-1 to 230-4 are set in the imaging region of the captured image 200.

The focus region setting unit 186 sets the focus detection regions 230-1 to 4 in association with the mask types ty1 to ty4. In the present embodiment, the focus detection regions 230-1 to 4 have the same range as the light amount detection regions 230-1 to 4. That is, the focus detection region 230-4 is set to include the evaluation frame 210-4 in the imaging region of the captured image 200 among the two evaluation frames 210-3 and 210-4 and the two evaluation frames 210-4 and 210-5 sandwiching the mask boundary 220-4 corresponding to the mask type ty4. Similarly, the focus detection region 230-3 is set to include the evaluation frames 210-3 and 210-5 in the imaging region of the captured image 200 among the two evaluation frames 210-2 and 210-3 and the two evaluation frames 210-5 and 210-6 sandwiching the mask boundary 220-3 corresponding to the mask type ty3.

Similarly, the focus detection region 230-2 is set to include the evaluation frames 210-2 and 210-6 in the imaging region of the captured image 200 among the two evaluation frames 210-1 and 210-2 and the two evaluation frames 210-6 and 210-7 sandwiching the mask boundary 220-2 corresponding to the mask type ty2. Similarly, the focus detection region 230-1 is set so as to include at least a part of the evaluation frames 210-1 and 210-7 in the imaging region of the captured image 200 among the two evaluation frames 210-0 and 210-1 and the two evaluation frames 210-7 and 210-8 sandwiching the mask boundary 220-1 corresponding to the mask type ty1. In this manner, the focus detection regions 230-1 to 230-4 are set in the imaging region of the captured image 200.

The evaluation value generation unit 192 calculates an evaluation value based on the pixel value of the captured image 200 in each of the evaluation frames 200-0 to 4, the evaluation frame 210-0, and the evaluation frames 210-1 to 8. For example, a pixel average value in the captured image 200 in the evaluation frame is set as the evaluation value. That is, the evaluation value is a value representing the luminance value in each of the evaluation frames 200-0 to 4, the evaluation frame 210-0, and the evaluation frames 210-1 to 8, that is, a value related to the luminance value.

The light reception value generation unit 194 calculates an evaluation value based on the pixel value of the captured image 200 in the frames of the light amount detection regions 230-1 to 4 according to the mask type.

For example, the average value of the pixel values in the captured image 200 within the frames in the light amount detection regions 230-1 to 4 is set as the evaluation value. That is, the evaluation value is a value related to the luminance value in the frames in the light amount detection regions 230-1 to 4.

The image quality value generation unit 196 calculates an evaluation value for AF based on the value of the captured image 200 in the frames of the focus regions 230-1 to 4 according to the mask type. For example, at least one of the contrast or the frequency component of the captured image 200 in the frames in the light amount detection regions 230-1 to 4 is set as the evaluation value. For example, the contrast value increases as the focus is adjusted. Furthermore, the frequency component on the high frequency side increases and the frequency component on the low frequency side decreases as the focus is adjusted. That is, this evaluation value is a value that increases as the contrast value increases.

Alternatively, the evaluation value is a value that increases as the ratio between the frequency component of the predetermined frequency band on the high frequency side and the frequency component of the predetermined frequency band on the low frequency side increases. As described above, the evaluation value for AF is a value that increases as the focuses in the frames of the light amount detection regions 230-1 to 4 are aligned.

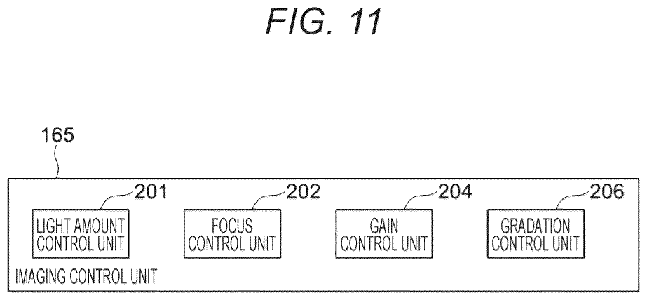

Here, the imaging control unit 165 will be described with reference to FIG. 11 while referring to FIGS. 3 and 7.

FIG. 11 is a block diagram illustrating a configuration example of the imaging control unit 165. As illustrated in FIG. 11, the imaging control unit 165 includes a light amount control unit 201, a focus control unit 202, a gain control unit 204, and a gradation control unit 206. Note that the imaging control unit 165 according to the present embodiment corresponds to a processing unit.

The light amount control unit 201 controls the light emission amount of the light source 172 via the control unit 171 such that, for example, the evaluation value generated by the light reception value generation unit 194 matches the AE target value. As described above, the control unit 171 controls the light emission amount of the light source 172 according to a light amount control signal output from the light amount control unit 201.

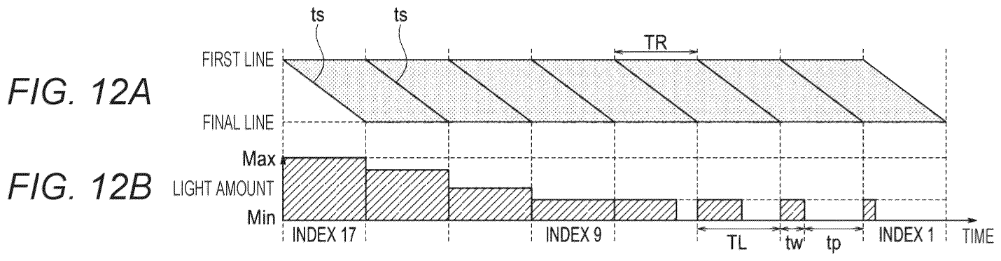

Note that, in FIG. 12, the light amount is described to be discretely changed for making the description simple, but the present invention is not limited thereto. That is, the light amount control unit 201 may continuously change the light emission amount of the light source 172.

FIG. 12 is a diagram illustrating a light emission adjustment example from a case where the target light amount of light emission of the light source device 53 is maximum (INDEX 17) to a case where the target light amount is minimum (INDEX 1). (a) of FIG. 12 illustrates an exposure state of the imaging element 521 (see FIG. 2), where the vertical axis represents a horizontal line of the imaging element 521, and the horizontal axis represents time. In (a) of FIG. 12, the uppermost line represents the uppermost horizontal line (that is, the first line), and the lowermost line represents the lowermost horizontal line (that is, the last line). A line (oblique line) indicated by a reference sign “ts” indicates a start timing for reading pixel data of each horizontal line regarding each captured image. (b) of FIG. 12 illustrates the light emission timing and the light amount of the light source 172 (see FIG. 3). The vertical axis in (b) of FIG. 12 indicates the light emission amount of the light source. The horizontal axis in (b) of FIG. 12 indicates time, indicates a continuous light emission time (that is, one pulse light emission time), and indicates an applied pulse (current drive pulse) of the current supplied to the light source.

Note that a value m of “INDEX” (where “m” is an integer of 1 to 17) represents a degree of a target light amount of light emission of the light source device 53, and the larger “m” is (the closer to “17”), the larger the target light amount is, and the smaller “m” is (the closer to“1”), the smaller the target light amount is.

Furthermore, “TR” indicates an exposure cycle of each horizontal line of the imaging element 521, and “TL” indicates a light emission cycle of the light source device 53. Furthermore, “tw” indicates a pulse width of light emission of the light source device 53 (that is, a pulse width of a current drive pulse supplied to the light source device 53). Furthermore, “tp” indicates an interval between adjacent light emission pulses of the light source device 53 (that is, a time interval between adjacent drive pulses of the current supplied to the light source device 53).

The AE target value is set such that, for example, the average luminance value of the region 210-4 (evaluation frame 4) becomes a constant value. Therefore, the light amount of the light source device 53 is controlled such that the evaluation values V230-1 to 4 in the light amount detection regions 230-1 to 4 (see FIG. 10) set according to the mask type become the AE target values.

As can be seen from above, in a case where the subject has low reflectance or in a case where the distance is long, the probability that a high index is selected is high, and in a case where the subject has high reflectance or in a case where the distance is short, the probability that a low index is selected is high (see (b) of FIG. 12).

Therefore, for example, when the end portion of the scope 101 approaches the subject in the body cavity or when an energy device or the like that emits strong light is used, the pulse width tw (eventually, the duty ratio) of the current drive pulse supplied to the light source is adjusted by PWM control in a state where the light emission amount from the light source is maintained at the minimum light amount (Min) (see (b) of FIG. 12).

The focus control unit 202 controls the position of the focus lens 511 such that, for example, the evaluation value generated by the image quality value generation unit 196 indicates the highest value. That is, the focus control unit 202 controls the position of the focus lens 511 via the camera head control unit 155 on the basis of the detection position of the lens position detection unit 512 and the evaluation value.

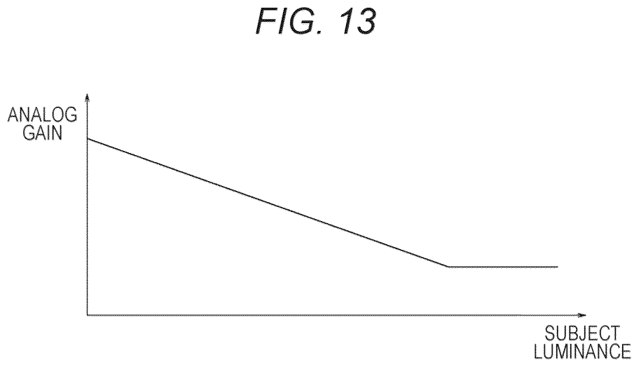

FIG. 13 is a diagram illustrating a relationship between the analog gain of the imaging unit 152 and the subject luminance generated by the light reception value generation unit 194. The horizontal axis represents the value of the subject luminance, and the vertical axis represents the value of the analog gain. As illustrated in FIG. 13, as the value of the subject luminance increases, the gain control unit 204 causes the signal processing unit 522 to execute processing of multiplying by an analog gain that decreases the electric signal (analog signal) from the imaging element 521. Note that the imaging unit 152 can also perform control processing by an electronic shutter with respect to an increase in the value of the subject luminance.

The gradation control unit 206 causes the image processing unit 163 to change the curve shape of the gamma curve (gradation conversion curve) of the endoscopic image on the basis of the evaluation value generated by the light reception value generation unit 194, for example, and generate the display image.

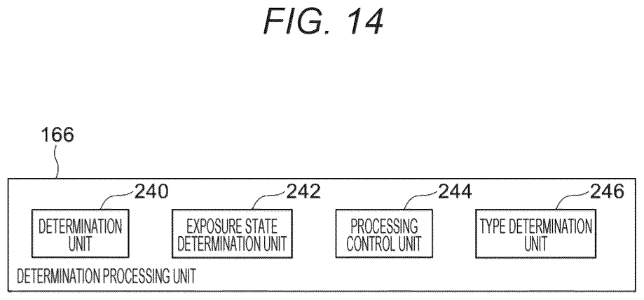

Here, details of the determination processing unit 166 will be described. FIG. 14 is a block diagram illustrating a configuration example of the determination processing unit 166. The determination processing unit 166 includes a determination unit 240, an exposure state determination unit 242, a processing control unit 244, and a type determination unit 246. The determination unit 240 executes first processing of determining whether or not the scope 101 is connected to the camera head 102.

Furthermore, the determination unit 240 determines the mask type in a case where it is determined that the scope 101 is connected to the camera head 102. Note that details of the determination unit 240 will be described later.

The exposure state determination unit 242 determines the exposure state on the basis of information of pixels within a predetermined range of the captured image. The exposure state determination unit 242 determines whether or not the type of the mask region can be correctly recognized. The time when the mask region cannot be correctly recognized is, for example, when the end portion of the scope 101 approaches the subject in the body cavity, when an energy device that emits strong light is used, or the like. Note that, in the present embodiment, a state in which the type of the mask region cannot be correctly recognized is referred to as a first state.

The processing control unit 244 causes the photometry unit 164 to execute processing with the mask type corresponding to the state determined by the exposure state determination unit 242 among the mask types determined by the determination unit 240. For example, in the first state, the processing control unit 244 causes the photometry unit 164 to execute processing with the mask type determined by the determination unit 240 immediately before the first state. Furthermore, the processing control unit 244 causes the imaging control unit 165 to execute control according to the state determined by the exposure state determination unit 242.

Note that details of the exposure state determination unit 242 and the processing control unit 244 will also be described later.

The type determination unit 246 determines the type of the scope 101 corresponding to the mask type determined by the determination unit 240. The mask type determined by the determination unit 240 corresponds to the type of the scope 101. Therefore, the type determination unit 246 sets the type of the scope 101 associated with the mask type determined by the determination unit 240 as the determination result. Note that the type determination unit 246 according to the present embodiment corresponds to an estimation unit.

### (First Processing of Determination Unit 240)

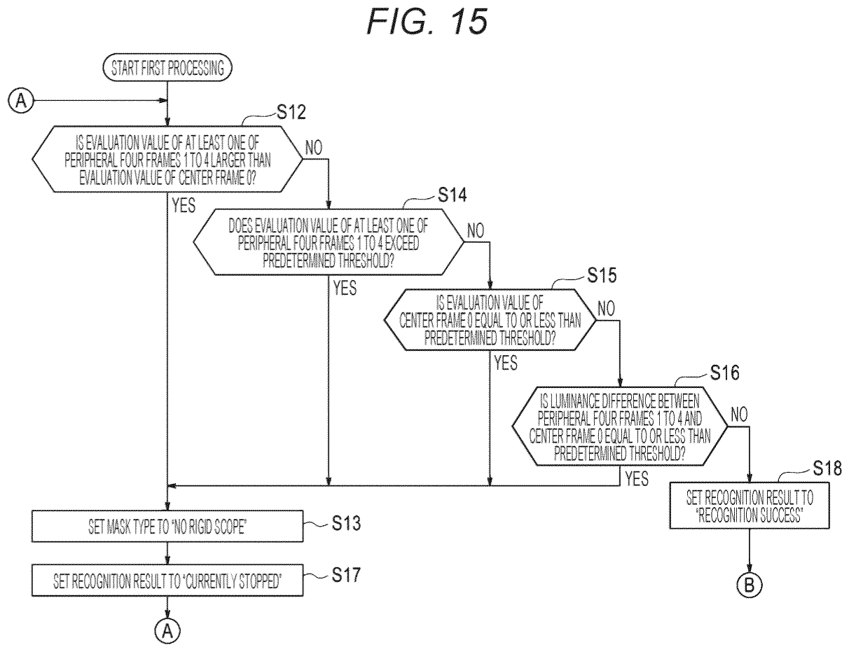

Here, a first processing example of the determination unit 240 will be described with reference to FIG. 15 while referring to FIG. 8. FIG. 15 is a flowchart of a first processing example. Here, the evaluation values of the evaluation frames 200-1 to 4 are set as the evaluation values V200-1 to 4, and the evaluation value of the evaluation frame 210-0 is set as the evaluation value V210-0.

The determination unit 240 determines whether or not any one of the evaluation values V200-1 to 4 is larger than the evaluation value V210-0 (step S12). In a case where the size is large (Yes in step S12), it is determined that the central portion of the captured image 200 is dark and the peripheral portion thereof is bright, and the absence of the scope 101 is set as a determination region of the storage unit 167 (see FIG. 3) (step S13).

On the other hand, in a case where all of the evaluation values V200-1 to 4 are equal to or less than the evaluation value V210-0 (No in step S12), the determination unit 240 determines whether or not any of the evaluation values V200-1 to 4 exceeds a predetermined threshold (first threshold) (step S14). In a case where it is determined to exceed (Yes in step S14), the process proceeds to step S13.

On the other hand, in a case where it is determined that all of the evaluation values V200-1 to 4 are equal to or less than a predetermined threshold, the determination unit 240 determines whether or not the evaluation value V210-0 is equal to or less than a predetermined threshold (second threshold) (step S15). In a case where it is equal to or less than the predetermined threshold (second threshold) (Yes in step S15), the process proceeds to step S13. On the other hand, in a case where it is determined that the evaluation value V210-0 exceeds the predetermined threshold (second threshold) (No in step S15), the determination unit 240 determines whether the luminance difference between the evaluation values V200-1 to 4 and the evaluation value V210-0 is equal to or less than a predetermined threshold (third threshold) (step S16). In a case where it is determined that the luminance difference is equal to or less than the predetermined threshold (Yes in step S16), the process proceeds to step S13. On the other hand, in a case where it is determined that the luminance difference is larger than the predetermined threshold (No in step S16), the recognition result is set to “recognition success” (step S18). After step S13, the process proceeds to step S17.

Then, in step S17, the determination unit 240 sets the recognition result as “currently stopped” in the storage unit 167 (see FIG. 3). Thereafter, the processing returns to step S12, and the above-described first processing is repeated. As described above, the determination unit 240 determines whether or not the scope 101 is connected to the camera head 102 according to the relationship between the brightness and darkness of the central portion and the peripheral portion of the captured image 200.

### (Second Processing of Determination Unit 240)

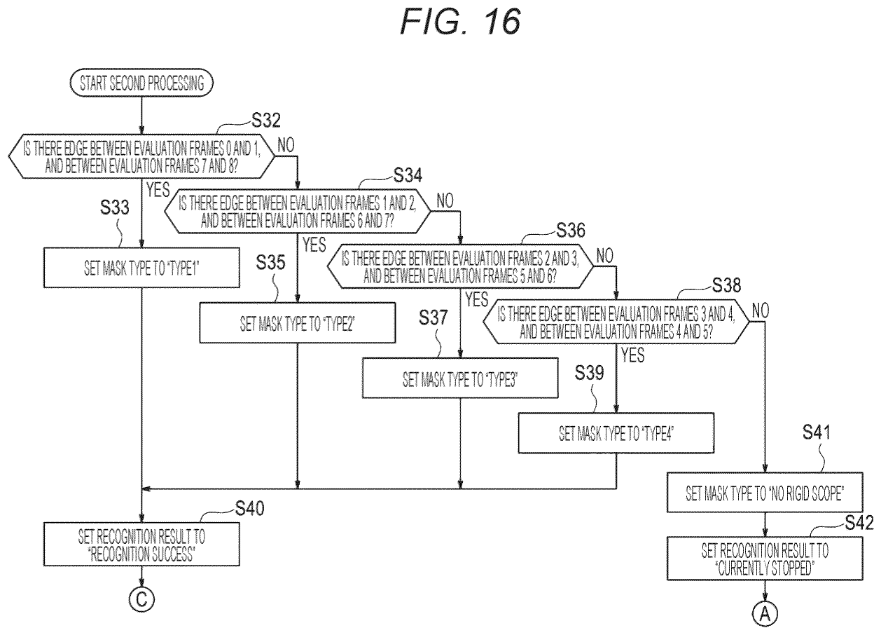

Here, a second processing example of the determination unit 240 will be described with reference to FIG. 15 while referring to FIG. 9. FIG. 16 is a flowchart of a second processing example. Here, the evaluation values of the evaluation frames 210-0 to 8 are set as evaluation values V210-0 to 8.

The determination unit 240 determines whether there is a mask boundary (edge) between the evaluation frame 0 and the evaluation frame 1 and whether there is a mask boundary 220-1 between the evaluation frame 7 and the evaluation frame 8 on the basis of the evaluation values V210-0 and V210-1 and the evaluation values V210-7 and V210-8 (step S32). For example, the difference between the evaluation value V210-0 and the evaluation value V210-1 and the difference between the evaluation value V210-7 and the evaluation value V210-8 are compared with a predetermined threshold (fourth threshold), and it is determined whether or not the difference exceeds the predetermined threshold, thereby determining whether or not there is a mask boundary 220-1 between these evaluation frames 210.

In a case where it is determined that there is the mask boundary 220-1 (Yes in step S32), the determination unit 240 sets the mask type as “TYPE1” in the storage unit 167 (step S33). On the other hand, in a case where it is determined that there is no mask boundary 220-1 (No in step S32), the determination unit 240 determines whether or not there is a mask boundary 220-2 between the evaluation frame 1 and the evaluation frame 2 and there is a mask boundary 220-2 between the evaluation frame 6 and the evaluation frame 7 on the basis of the evaluation value V210-1, the evaluation value V210-2, the evaluation value V210-6, and the evaluation value V210-7 (step S34).

In a case where it is determined that there is the mask boundary 220-2 (Yes in step S34), the determination unit 240 sets the mask type as “TYPE2” in the storage unit 167 (step S35). On the other hand, in a case where it is determined that there is no mask boundary 220-2 (No in step S34), the determination unit 240 determines whether or not there is a mask boundary 220-3 between the evaluation frame 2 and the evaluation frame 3 and there is a mask boundary 220-3 between the evaluation frame 5 and the evaluation frame 6 on the basis of the evaluation value V210-2, the evaluation value V210-3, the evaluation value V210-5, and the evaluation value V210-6 (step S36).

In a case where it is determined that there is the mask boundary 220-3 (Yes in step S36), the determination unit 240 sets the mask type as “TYPE3” in the storage unit 167 (step S37). On the other hand, in a case where it is determined that there is no mask boundary 220-3 (No in step S36), the determination unit 240 determines whether or not there is a mask boundary 220-4 between the evaluation frame 3 and the evaluation frame 4 and there is a mask boundary 220-4 between the evaluation frame 4 and the evaluation frame 6 on the basis of the evaluation value V210-3, the evaluation value V210-4, the evaluation value V210-4, and the evaluation value V210-5 (step S38).

In a case where it is determined that there is the mask boundary 220-4 (Yes in step S38), the determination unit 240 sets the mask type as “TYPE4” in the storage unit 167 (step S39). On the other hand, in a case where it is determined that there is no mask boundary 220-4 (No in step S38), the determination unit 240 sets the mask type as “no rigid scope (scope)” in the storage unit 167 (step S41), and sets the recognition result as “currently stopped” in the storage unit 167 (step S42). Thereafter, the process returns to step S12 in FIG. 14, and the above-described first processing is executed. On the other hand, after steps S32, S34, S36, and S38, the determination unit 240 sets the recognition result in the storage unit 167 as “recognition success” (step S40). In this manner, the determination unit 240 determines the position of the mask boundary based on the values of the evaluation values V210-0 to V210-8.

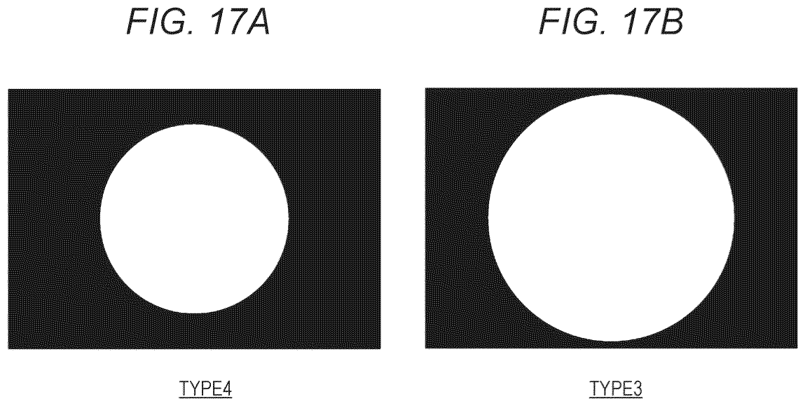

Here, processing examples of the exposure state determination unit 242 and the processing control unit 244 will be described with reference to FIGS. 17 to 20.

### (Determination Condition of First State)

Here, a determination processing example of the first state in which the mask type cannot be correctly recognized will be described. The exposure state determination unit 242 determines that it is the first state that cannot be correctly recognized in a case of a combination of the following condition 1 and at least one of conditions 2 to 3. In particular, in a case where all of the following conditions 1 to 4 are satisfied, it is determined that the first state cannot be correctly recognized with the highest probability. Therefore, in the following embodiment, an example in a case where all of the following conditions 1 to 4 are satisfied will be described.

In a case where the first state is determined, the exposure state determination unit 242 turns on the first mode and stores the current state in the storage unit 167 (see FIG. 3). On the other hand, in a case where the first state is not determined, the exposure state determination unit 242 turns off the first mode and stores the current state in the storage unit 167 (see FIG. 3).

The condition 1 is that the mask type determined by the determination unit 240 changes. The target combinations of the condition 1 may be, for example, any of changes from the mask type ty4 to the type ty3, from the mask type ty4 to the type ty2, from the mask type ty4 to the type ty1, and from the mask type ty4 to no connection.

Similarly, there is a case of any of changes from the mask type ty3 to the type ty2, from the mask type ty3 to the type ty1, and from the mask type ty3 to no connection. Similarly, there is a case of any of changes from the mask type ty2 to the type ty1 and from the mask type ty2 to no connection. Similarly, there is a case of a change from the mask type ty1 to no connection. In this manner, the determination condition of the first state is a case where the mask type has changed. That is, it is assumed that the mask types determined by the determination unit 240 are different in time series.

The condition 2 is that the pixel value average, which is the evaluation value V210-4 of the evaluation frame 210-4 (see FIG. 9) in the central portion of the captured image 200, is larger than a value obtained by adding the discrimination threshold 2 to the AE target value. That is, the condition of evaluation value V210-4>discrimination threshold 2+AE target value is satisfied. As described above, the determination condition of the first state is a case where the evaluation value based on the pixel value in the evaluation frame 210-4 in the central portion of the captured image 200 is larger than a value obtained by adding the discrimination threshold 2 to the AE target value as a predetermined value.

The condition 3 is that the evaluation value in the evaluation frame on the imaging region side used by the determination unit 240 to determine the mask type is equal to or more than the discrimination threshold 1 and is sufficiently lower than the value of the evaluation value V210-4. For example, it is 5% or less of the value of the evaluation value V210-4. Here, the evaluation values corresponding to the evaluation frames 210-0 to 8 are set as evaluation values V210-0 to 8.

For example, in a case where there is a change from the mask type ty4 to the type ty3, from the mask type ty4 to the type ty3, from the mask type ty4 to the type ty1, or from the mask type ty4 to no connection, the evaluation values V210-3 and V210-5 are equal to or larger than the discrimination threshold 1 and are values sufficiently lower than the value of the evaluation value V210-4. For example, the evaluation values V210-3 and V210-5 are 5% or less of the value of the evaluation value V210-4.

Similarly, in a case where any of changes from the mask type ty3 to the type ty2, from the mask type ty3 to the type ty1, and from the mask type ty3 to no connection has occurred, the evaluation values V210-2 and V210-6 of the evaluation frame 210-2 and the evaluation frame 210-6 (see FIG. 9) are equal to or more than the discrimination threshold 1 and are sufficiently lower than the value of the evaluation value V210-4. For example, the evaluation values V210-2 and V210-6 are 5% or less of the value of the evaluation value V210-4.

Similarly, in a case where there is any of changes from the mask type ty2 to the type ty1 and from the mask type ty2 to no connection, the evaluation values V210-1 and V210-7 of the evaluation frame 210-1 and the evaluation frame 210-7 (see FIG. 9) are values equal to or larger than the discrimination threshold 1 and sufficiently lower than the value of the evaluation value V210-4. For example, the evaluation values V210-1 and V210-7 are 5% or less of the value of the evaluation value V210-4.

Similarly, in a case where there is a change from the mask type ty1 to no connection, the evaluation values V210-0 and V210-8 of the evaluation frame 210-0 and the evaluation frame 210-8 (see FIG. 9) are equal to or more than the discrimination threshold 1, and are sufficiently lower than the value of the evaluation value V210-4. For example, the evaluation values V210-1 and V210-7 are 5% or less of the value of the evaluation value V210-4. As described above, the determination condition of the first state is a case where the evaluation value in the evaluation frame on the imaging region side used for the mask type determination is greater than or equal to the discrimination threshold 1 and less than or equal to 5%, which is an example of a predetermined ratio, as compared with the value of the evaluation value V210-4.

The condition 4 is a state in which the evaluation values of the light amount detection regions 230-1 to 4 (see FIG. 10) according to the mask type are within AE target value±predetermined range. The predetermined range is, for example, ±5% of the AE target value. As described above, the exposure state determination unit 242 determines that the exposure state is the first state in which the type of the mask region cannot be correctly recognized in a case where at least the first mask type determined on the basis of the captured image and the second mask type determined on the basis of the image acquired before imaging are different and the evaluation value based on the pixel value in the evaluation frame 4 in the central portion of the captured image is larger than a predetermined value. As described above, the determination condition of the first state is a case where the evaluation values of the light amount detection regions 230-1 to 4 (see FIG. 10) are within AE target value±predetermined range (for example, 10% of the AE target value).

### (Condition for Canceling First State)

When the following condition is satisfied after determining as the first state, the exposure state determination unit 242 determines as the normal state (second state) in which the type of the mask region can be correctly recognized. That is, it is determined that the first state has been canceled. In at least any one case of the following conditions 5 to 8, it is determined as a normal state that can be correctly recognized. In particular, in a case where all of the following conditions 5 to 8 are satisfied, it is determined as a normal state in which correct recognition can be performed with the highest probability. Therefore, in the following embodiment, an example in a case where all of the following conditions 5 to 8 are satisfied will be described.

The condition 5 is that the pixel value average, which is the evaluation value V210-4 of the evaluation frame 210-4 (see FIG. 9) in the central portion of the captured image 200, is smaller than a value obtained by adding the discrimination threshold 2 to the AE target value. That is, the condition of evaluation value V210-4<discrimination threshold 2+AE target value is satisfied. As described above, the condition for canceling the first state is a case where the evaluation value based on the pixel value in the evaluation frame 210-4 in the central portion of the captured image 200 is smaller than a value obtained by adding the discrimination threshold 2 to the AE target value as the predetermined value.

The condition 6 is that the evaluation value in the evaluation frame on the mask region side used by the determination unit 240 to determine the mask type is equal to or less than the discrimination threshold 1.

More specifically, there is a case where the mask type stored in the storage unit 167 (see FIG. 3) is the type ty4, that is, a case where the mask type before the determination as the first state is the type ty4. In this case, when the evaluation values of the evaluation frames 3 and 5 become equal to or less than the threshold 1, the exposure state determination unit 242 determines that the condition 6 is satisfied.

Similarly, there is a case where the mask type stored in the storage unit 167 (see FIG. 3) is the type ty3, that is, a case where the mask type before the determination as the first state is the type ty3. In this case, when the evaluation values of the evaluation frames 2 and 6 become equal to or less than the threshold 1, the exposure state determination unit 242 determines that the condition 6 is satisfied.

Similarly, there is a case where the mask type stored in the storage unit 167 (see FIG. 3) is the type ty2, that is, a case where the mask type before the determination as the first state is the type ty2. In this case, when the evaluation values of the evaluation frames 1 and 7 become equal to or less than the threshold 1, it is determined that the condition 6 is satisfied.

Similarly, there is a case where the mask type stored in the storage unit 167 (see FIG. 3) is the type ty1, that is, a case where the mask type before the determination as the first state is the type ty1. In this case, when the evaluation values of the evaluation frames 0 and 8 become equal to or less than the threshold 1, the exposure state determination unit 242 determines that the condition 6 is satisfied.

The condition 7 is that the state of the condition 6 is maintained for a predetermined period. That is, the condition is that the measurement environment is stable.

The predetermined period can be set to any period including 0.

In the condition 8, it is assumed that the evaluation value used for the light amount control among the evaluation values V230-1 to 4 is maintained within AE target value±predetermined range. That is, the condition is that the measurement environment is stable.

The period during which the state within the predetermined range is maintained can be set to any period including 0.

### (Processing Example in First State of Processing Control Unit 244)

In a case where the exposure state determination unit 242 determines that the exposure state is in the first state, the processing control unit 244 executes the following processing.

The processing control unit 244 outputs a control processing signal instructing processing with the mask type determined by the determination unit 240 most recently before the first state, to the light reception value generation unit 194 and the image quality value generation unit 196 of the generation unit 190 (see FIG. 7). Therefore, a decrease in processing accuracy of the imaging control unit 165 is suppressed. The imaging control unit 165 according to the present embodiment corresponds to a processing unit.

Furthermore, in a case where the exposure state determination unit 242 determines that the light amount is in a proximity saturation state, the processing control unit 244 executes control for reducing the light amount of the light source 172 (see FIG. 3) by PWM control at the index of 8 or less with respect to the light amount control unit 201 (see FIG. 11). Therefore, the first state can be canceled in a shorter time.

Therefore, the operator (doctor) 3 can observe the image in a shorter time even in the first state.

Moreover, the processing control unit 244 controls the focus control unit 202 (see FIG. 11) and the gain control unit 204 (see FIG. 11) to stop the control. This makes it possible to suppress malfunction due to the first state.

Moreover, the processing control unit 244 causes the gradation control unit 206 (see FIG. 11) to change the gamma curve to display a dynamic range of a wide range.

Therefore, the operator (doctor) 3 can observe the image even in the first state.

Furthermore, the processing control unit 244 causes the display device 52 to display an image in a display mode indicating the first state. At this time, the processing control unit 244 can also generate a voice indicating the first state when causing the display device 52 to display. Therefore, the operator (doctor) 3 can correct the position of the scope 101.

Next, a processing example of the exposure state determination unit 242 will be described more specifically with reference to FIGS. 17 to 20. Here, a processing example of the exposure state determination unit 242 will be described using an example of erroneously determining the mask type ty4 as the mask type ty3. FIGS. 17A and 17B are diagrams illustrating a mask type ty4 and a mask type ty3. FIG. 17A illustrates the mask type ty4, and FIG. 17B illustrates the mask type ty3. The imaging region of the mask type ty3 is configured to be larger than the imaging region of the mask type ty4.

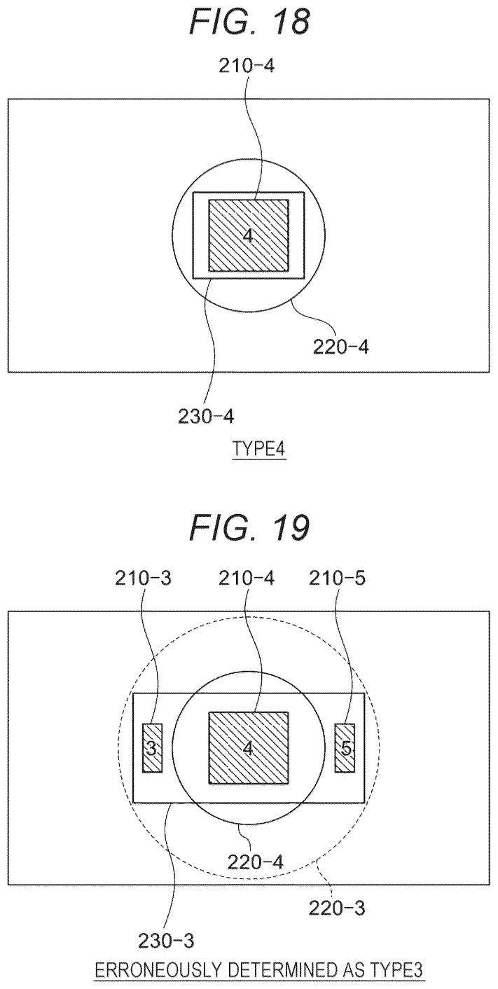

FIG. 18 is a diagram illustrating an example of an evaluation frame of the mask type ty4. The mask boundary 220-4 of the mask type ty4, the evaluation frame 210-4, and the light amount detection region 230-4 corresponding to the mask type ty4 are illustrated. The evaluation value based on the light amount detection region 230-4 is set to V230-4, and the evaluation value based on the evaluation frame 210-4 is set to V210-4. These evaluation values are pixel value averages.

FIG. 19 is a diagram illustrating an example of an evaluation frame of the mask type ty3. FIG. 19 illustrates an example of erroneous determination as the mask type ty3. The mask boundary 220-3 of the mask type ty3, the mask boundary 220-4 of the mask type ty4, the evaluation frames 210-3 to 210-5, and the light amount detection region 230-3 corresponding to the mask type ty3 are illustrated. An evaluation value based on the light amount detection region 230-3 is set to V230-3, and an evaluation value based on the evaluation frame 210-4 is set to V210-4. Furthermore, the evaluation values based on the evaluation frames 210-3 and 210-5 are denoted by V210-3 and V210-5. These evaluation values are pixel value averages.

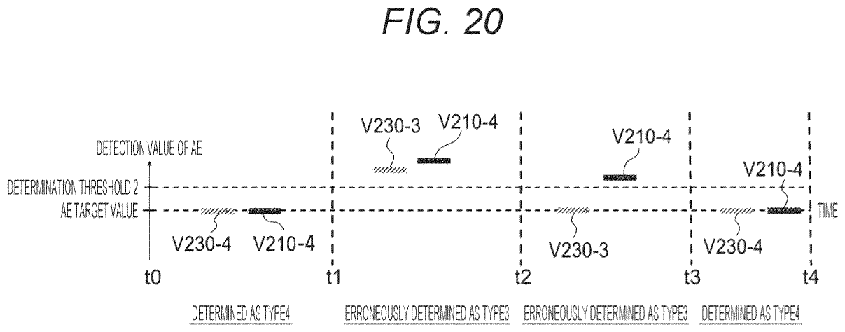

FIG. 20 is a state transition diagram illustrating a determination result of a mask type. The horizontal axis represents time. The evaluation value V230-4, the evaluation value V230-3, and the evaluation value V210-4 are the values described in FIGS. 18 and 19.

With reference to FIG. 18, the exposure state determination unit 242 determines that the exposure state is the normal state between the times t0 and t1. That is, it is a period that is not in the first state. In the normal state, the processing control unit 244 outputs a control processing signal instructing processing with the mask type determined by the determination unit 240 to the light reception value generation unit 194 and the image quality value generation unit 196 of the photometry unit 164 (see FIG. 7). Therefore, the light amount control unit 201 controls the light amount of the light source 172 (see FIG. 3) so that the evaluation value V230-4 becomes the AE target value. In this case, the evaluation value V210-4 is also a value close to the AE target value.

On the other hand, between times t1 and t2, for example, a first state in which the reflected light from the subject rapidly increases is illustrated. Therefore, the imaging light leaking from the imaging region surrounded by the mask boundary 220-4 reaches the evaluation frames 210-3 and 210-5, and the evaluation value V210-3 and the evaluation value V210-5 have values equivalent to the evaluation value V210-4. Therefore, it is erroneously determined that the mask boundary 220-3 is present between the evaluation frames 2 and 3 and the mask boundary 220-3 (see FIG. 10) is present between the evaluation frames 5 and 6. That is, the mask type is erroneously determined as the mask type ty3.

Since it is in the normal state, the processing control unit 244 outputs a control processing signal instructing processing with the mask type ty3 determined by the determination unit 240 to the light reception value generation unit 194 of the photometry unit 164 (see FIG. 7). Therefore, as illustrated in FIG. 19, the light reception value generation unit 194 (see FIG. 7) outputs the evaluation value V230-3 based on the incorrect light amount detection region 230-3 to the light amount control unit 201. Therefore, control is started in which the evaluation value V230-3 based on the incorrect light amount detection region 230-3 becomes the AE target value. Furthermore, since the mask type has been changed, the exposure state determination unit 242 starts determination of the conditions 1 to 4. During this period, because the condition 4 is not satisfied, the exposure state determination unit 242 maintains the normal state.

Between times t2 and t3, it is erroneously determined as the type ty3, and as illustrated in FIG. 18, the light amount control unit 201 controls the evaluation value V230-3 to be the AE target value on the basis of the value of the evaluation value V230-3. Since the evaluation value V230-3 is calculated including the mask region, the image average value that is the control value is smaller than the average value of the imaging region.

Therefore, when the light amount control unit 201 controls the evaluation value V230-3 to be the AE target value, the average value of the imaging region indicates a high pixel (corresponding to high luminance).

Therefore, the evaluation value V230-3 is in a state of being controlled to be the AE target value, but the evaluation value V210-4 exceeds the threshold 2.

In this period, since the conditions 1 to 4 are satisfied, the exposure state determination unit 242 determines the first state. That is, the mask type is changed from the mask type ty4 to the type ty3 (condition 1). Further, the evaluation value V210-4 is larger than a value obtained by adding the discrimination threshold 2 to the AE target value (condition 2).

Since the light amount control unit 201 controls the light amount to be larger than usual due to erroneous determination, the evaluation values V210-3 and V210-5 become equal to or larger than the discrimination threshold 1. Therefore, the evaluation values V210-3 and V210-5 are equal to or larger than the discrimination threshold 1 and are sufficiently lower than the value of the evaluation value V210-4 (condition 3). At this time, the processing control unit 244 controls the light amount control unit 201 (see FIG. 11) to reduce the light amount of the light source 172 (see FIG. 3) by PWM control at an index of 8 or less. Therefore, the evaluation value V230-3 is controlled to be the AE target value (condition 4). Thus, the conditions 1 to 4 are satisfied.

Therefore, the processing control unit 244 outputs a control processing signal instructing processing with the mask type ty4 determined by the determination unit 240 most recently before the first state is obtained to the light reception value generation unit 194 and the image quality value generation unit 196 of the photometry unit 164 (see FIG. 7). The mask type ty4 most recently determined by the determination unit 240 is stored in the storage unit 167 (see FIG. 3). At this time, the processing control unit 244 can cause the light amount control unit 201 (see FIG. 11) to execute control for reducing the light amount of the light source 172 (see FIG. 3) by PWM control at an index of 8 or less.

Between times t3 and t4, the processing in the mask type ty4 is executed, and the light amount is further reduced.

Therefore, the evaluation value V230-4 is controlled to be the AE target value by the light amount control unit 201. In this case, the evaluation value V210-4 is also a value close to the AE target value.

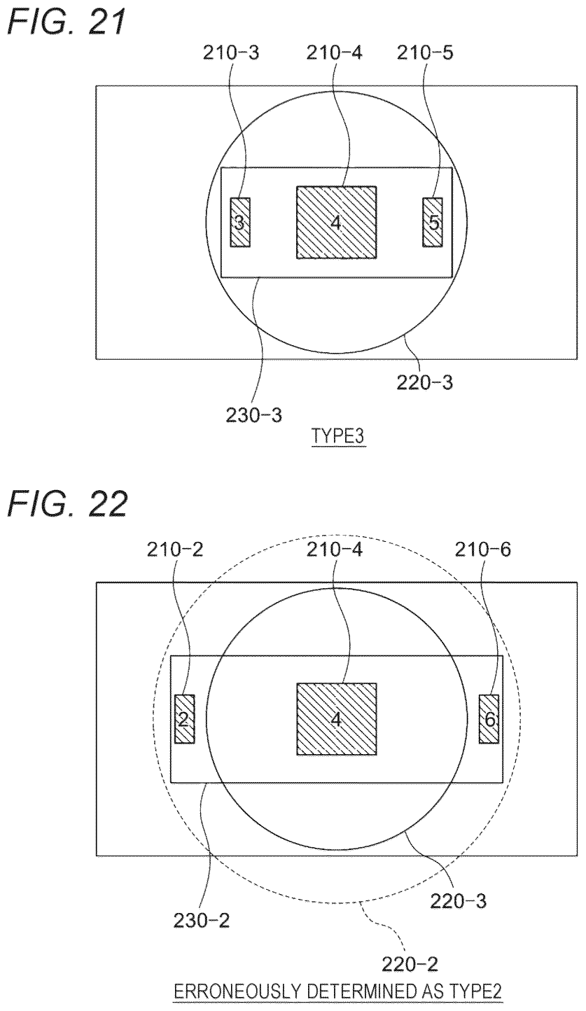

Next, an example of a state in which the first mode transitions from the OFF state to the ON state will be described with reference to FIGS. 21 to 24. Here, an example of erroneously determining the mask type ty3 as the mask type ty2 will be described.

FIG. 21 is a diagram illustrating an example of an evaluation frame of the mask type ty3. The mask boundary 220-3 of the mask type ty3, the evaluation frames 210-3 to 210-5, and the light amount detection region 230-3 corresponding to the mask type ty3 are illustrated. An evaluation value based on the light amount detection region 230-3 is set to V230-3, and an evaluation value based on the evaluation frame 210-4 is set to V210-4.

Furthermore, the evaluation values based on the evaluation frames 210-3 and 210-5 are denoted by V210-3 and V210-5. These evaluation values are pixel value averages.

FIG. 22 is a diagram illustrating an example of an evaluation frame of the mask type ty2. The mask boundary 220-3 of the mask type ty3, the mask boundary 220-2 of the mask type ty2, the evaluation frames 210-2, 210-4, and 210-6, and the light amount detection region 230-2 corresponding to the mask type ty2 are illustrated. The evaluation value based on the light amount detection region 230-2 is set to V230-2, and the evaluation values based on the evaluation frames 210-2, 210-4, and 210-6 are set to V210-2, V210-4, and V210-6. These evaluation values are pixel value averages.

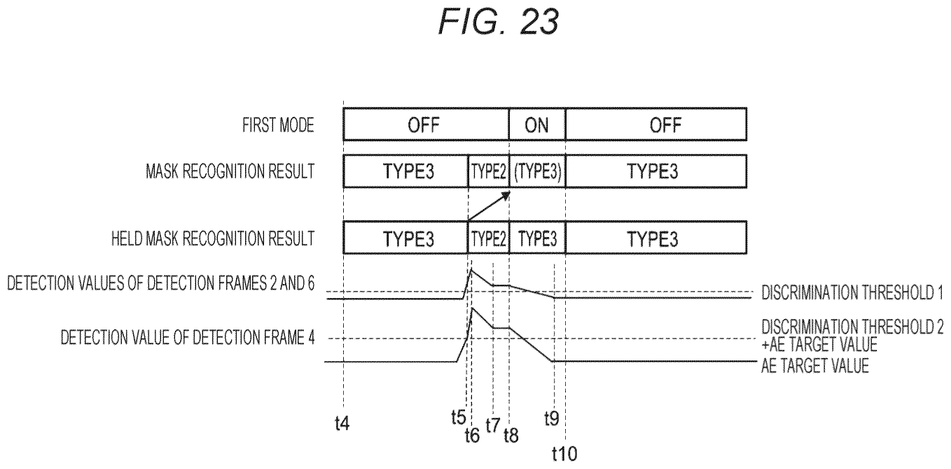

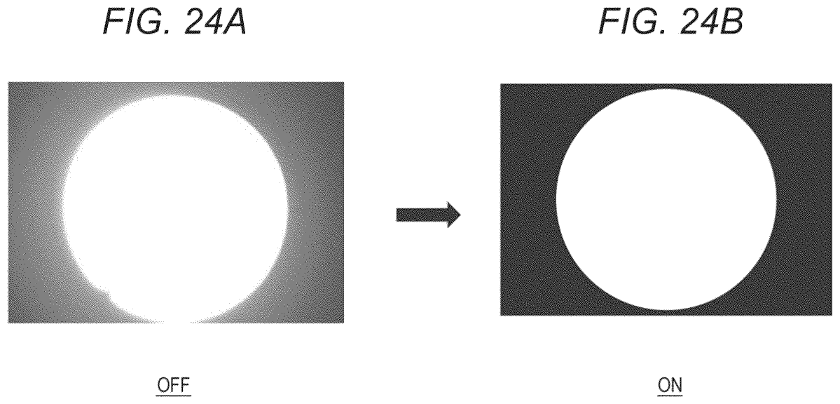

FIG. 23 is a time chart illustrating the transition of the first mode from the OFF state to the ON state. The ON and OFF states of the first mode, the mask determination result, the held mask determination result, the detection values (evaluation values) of the evaluation frames 2 and 6, and the detection value (evaluation value) of the evaluation frame 4 are illustrated in order from the top. FIG. 24 is a diagram illustrating an example of a captured image in the first state. (a) of FIG. 24 is an example of a captured image at time t6 (see FIG. 23), and (b) of FIG. 24 is an example of a captured image after time t9 (see FIG. 23) at which the evaluation value of the evaluation frame 4 converges to the AE target value after the evaluation value fluctuates.

As illustrated in FIG. 23, the control processing is started from time t4. At time t4, since the first mode is in the OFF state, the processing control unit 244 executes the processing with the mask type determined by the determination unit 240. At time t4, the determination unit 240 stores the mask type ty3 in the storage unit 167 (see FIG. 3) as a determination result.

The evaluation values V210-2 and V210-6 are less than the discrimination threshold 1. Furthermore, the evaluation value V210-4 is less than discrimination threshold 2+AE target value, which is less than the discrimination threshold 1. Therefore, since the conditions 1 to 4 are not satisfied, the exposure state determination unit 242 maintains the first mode in the OFF state. Subsequently, for example, proximity of the scope 101 occurs, the amount of light incident on the scope 101 rapidly increases, the light amount reduction control cannot catch up, and the values of the evaluation values V210-2 and V210-6 increase.

At time t5, the determination unit 240 erroneously determines the mask type as a type ty2. Therefore, the light amount control unit 201 (see FIG. 11) continues the light amount reduction control with the evaluation value V230-2 based on the incorrect light amount detection region 230-2. Furthermore, the mask type determined by the determination unit 240 is changed from the mask type ty3 to the mask type ty2, and satisfies the condition 1.

At this time, the processing control unit 244 controls the light amount control unit 201 (see FIG. 11) to reduce the light amount of the light source 172 (see FIG. 3) by PWM control at an index of 8 or less. Therefore, the amount of light incident on the scope 101 turns to decrease at time t6.

At time t7, the values of the evaluation values V210-2 and V210-6, and the value of the evaluation value V210-4 are in a stable state. Therefore, the condition 4 is satisfied.

Since the light amount control unit 201 uses the evaluation value V230-2 based on the incorrect light amount detection region 230-2, the evaluation value V210-4 is stabilized in a state of exceeding discrimination threshold 2+AE target value. Therefore, the condition 2 is satisfied.

Similarly, since the light amount control unit 201 uses the evaluation value V230-2 based on the incorrect light amount detection region 230-2, the values of the evaluation values V210-2 and V210-6 are stabilized in a state of exceeding the discrimination threshold 1.

Therefore, the condition 3 is satisfied.

At time t8, the exposure state determination unit 242 determines that the conditions 1 to 4 are satisfied.

Therefore, the first mode transitions to the ON state.

Since the first mode has transitioned to the ON state, the determination unit 240 executes the control processing with the mask type ty3 stored in the storage unit 167 (see FIG. 3).

Since the light amount control unit 201 uses the evaluation value V230-3 based on the correct light amount detection region 230-3, the evaluation value is controlled to be the AE target value. Therefore, at time t9, the evaluation value V210-4 also approaches the AE target value and is stabilized. As described above, the evaluation value V210-4 of the evaluation frame 210-4 (see FIG. 9) in the central portion of the captured image 200 becomes smaller than the value obtained by adding the discrimination threshold 2 to the AE target value, and a state in which the condition 5 and the condition 8 are satisfied is obtained.

Similarly, at time t9, the values of the evaluation values V210-2 and V210-6 become less than the discrimination threshold 1 and become stable. In a case where the mask type before being determined as the first state is the type ty3, and when the values of the evaluation values V210-2 and V210-6 become equal to or less than the discrimination threshold 1, the exposure state determination unit 242 determines that the condition 6 is satisfied. Therefore, the conditions 6 and 7 are satisfied.

At time t10, the exposure state determination unit 242 determines that the conditions 5 to 8 are satisfied.

Therefore, the exposure state determination unit 242 determines that the first state has been canceled, and sets the first mode to be in the OFF state. That is, the control shifts to a state in which the mask type can be correctly recognized.

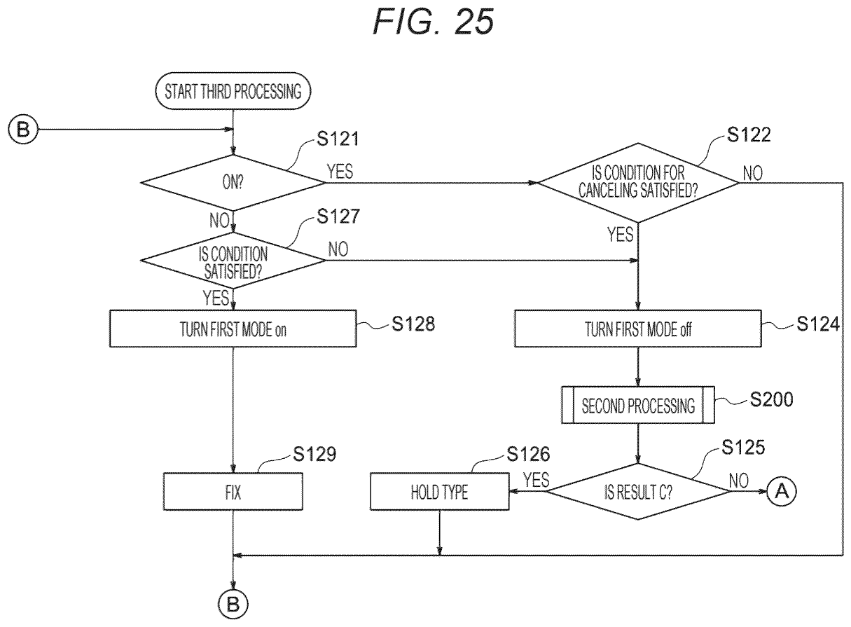

FIG. 25 is a flowchart illustrating a processing example after the determination processing of FIG. 15. When the determination unit 240 determines that the scope 101 is present, the processing control unit 244 determines whether or not the first mode stored in the storage unit 167 (see FIG. 3) is turned on (step S121). When it is determined that the processing control unit 244 is turned on (y in step S121), the exposure state determination unit 242 determines whether or not a condition for canceling the first state is satisfied (step S122). In the case of not being satisfied (n in step S122), the processing from step S121 is repeated.

On the other hand, in a case where the exposure state determination unit 242 determines that the condition for canceling the first state is satisfied (y in step S122), the exposure state determination unit 242 turns off the first mode and stores the first mode in the storage unit 167 (step S124). Subsequently, the determination unit 240 executes the second processing illustrated in FIG. 16 (step S200).

The determination unit 240 determines whether or not the processing result of the second processing is C (step S125). In a case where it is determined that the result is not C (n in step S125), the first processing illustrated in FIG. 15 is executed. On the other hand, in a case where it is determined that the result is C (y in step S125), the mask type is stored in the storage unit 167 (step S126), and the processing from step S121 is repeated.

On the other hand, when the processing control unit 244 is determined to be off (n in step S121), the exposure state determination unit 242 executes the determination processing of the first state (step S127). In this case, for example, the determination processing of the conditions 1 to 4 of the first state may be repeated 10 times.

In a case where it is determined that the current state is the first state (y in step S127), the exposure state determination unit 242 turns on the first mode and stores the first mode in the storage unit 167 (step S128). The processing control unit 244 executes the control processing while fixing the mask type stored in the storage unit 167 (step S129), and repeats the processing from step S121.

As described above, according to the present embodiment, the exposure state determination unit 242 determines the first state on the basis of the information of the pixel values in the predetermined range 210-4 of the captured image 200, and the processing control unit 244 causes the imaging control unit 165 or the like to execute processing with the mask type according to the state determined by the exposure state determination unit 242 among the mask types determined by the determination unit 240. Therefore, it is possible to suppress processing with a mask type having a high possibility of erroneous determination, and it is possible to suppress a decrease in processing accuracy of the imaging control unit 165.

It should be noted that the embodiments and modifications disclosed herein are merely illustrative in all respects and are not to be construed as limiting. The above-described embodiments and modifications can be omitted, replaced, and changed in various forms without departing from the scope and spirit of the appended claims. For example, the above-described embodiments and modifications may be combined in whole or in part, and embodiments other than the above-described embodiments and modifications may be combined with the above-described embodiments or modifications. Furthermore, the effects of the present disclosure described in the present specification are merely examples, and other effects may be provided.

Note that the present technology can have the following configurations.

(1)

A medical image capturing system that captures imaging light including a subject image obtained by irradiating a body cavity with illumination light from a light source unit, the medical image capturing system including:
an imaging unit that captures, via a scope, a captured image including an imaging region where the imaging light reaches an imaging surface and a mask region where the imaging light does not reach the imaging surface;
an exposure state determination unit that determines an exposure state on the basis of information of pixels within a predetermined range of the captured image; and
a processing unit that executes processing by setting a type of the mask region as a first mask type determined on the basis of the captured image or a second mask type determined on the basis of an image acquired before the imaging according to the exposure state.
   (2)

The medical image capturing system according to (1), in which the exposure state determination unit determines whether or not it is a state in which a type of the mask region can be correctly recognized.

(3)

The medical image capturing system according to (1) or (2), in which in a case where the exposure state determination unit determines that the exposure state is a state in which the type of the mask region can be correctly recognized, the processing unit performs processing based on the first mask type.

(4)

The medical image capturing system according to any one of (1) to (3), in which in a case where the exposure state determination unit determines that the exposure state is a state in which a type of the mask region cannot be correctly recognized, the processing unit performs processing based on the second mask type.

(5)

The medical image capturing system according to any one of (1) to (4), in which the processing unit controls a light emission amount of the light source unit according to a detection region corresponding to a type of the mask region.

(6)

The medical image capturing system according to (5), in which the processing unit reduces the light emission amount by PWM control.

(7)

The medical image capturing system according to any one of (1) to (6), in which
the imaging unit captures the captured image via a focus lens, and
the processing unit controls a focus of the focus lens according to a type of the mask region.
   (8)

The medical image capturing system according to (7), in which the processing unit stops focus control of the focus lens in a case where the exposure state determination unit determines that the exposure state is a state in which a type of the mask region cannot be correctly recognized.

(9)

The medical image capturing system according to (1), in which the processing unit controls a gain of a signal output from the imaging unit according to a type of the mask region.

(10)

The medical image capturing system according to (1), in which the processing unit executes gradation conversion of the captured image according to a type of the mask region.

(11)

The medical image capturing system according to (10), in which in a case where the exposure state determination unit determines that the exposed state is a state in which a type of the mask region cannot be correctly recognized, the processing unit executes gradation conversion for widening a dynamic range more than that at a time of the determination.

(12)

The medical image capturing system according to any one of (1) to (10), in which the exposure state determination unit determines that the exposure state is a state in which a type of the mask region cannot be correctly recognized at least in a case where the first mask type and the second mask type are different and an evaluation value based on a pixel value in an evaluation frame in a central portion of the captured image is larger than a predetermined value.

(13)

The medical image capturing system according to any one of (1) to (12), further including: a setting unit that sets a plurality of evaluation frames such that a mask boundary between the mask region of the first mask type and the imaging region is between the plurality of evaluation frames.

(14)

The medical image capturing system according to (13), in which the exposure state determination unit sets, as a condition that the exposure state is a state in which a type of the mask region cannot be correctly recognized, that an evaluation value in the evaluation frame on the imaging region side of the two evaluation frames sandwiching the mask boundary of the first mask type is a predetermined value or more under control of the processing unit.

(15)

The medical image capturing system according to (14), in which the exposure state determination unit sets, as a condition that the exposure state is a state in which a type of the mask region cannot be correctly recognized, that a pixel value average in an evaluation frame in a central portion of the captured image is a predetermined value or more.

(16)

The medical image capturing system according to (13), in which the exposure state determination unit sets, as a condition that the exposure state is a state in which a type of the mask region can be correctly recognized, that an evaluation value in the evaluation frame on the mask region side of the two evaluation frames sandwiching the mask boundary of the first mask type is a predetermined value or less under control of the processing unit.

(17)

The medical image capturing system according to (13), in which the setting unit sets the plurality of evaluation frames to be point-symmetric with respect to a corresponding point of an optical axis of the scope.

(18)

The medical image capturing system according to any one of (1) to (17), further including:
an estimation unit that estimates a type of the scope on the basis of a type of the mask region.
   (19)

A method of capturing a medical image that captures imaging light including a subject image obtained by irradiating a body cavity with illumination light from a light source unit, the method including:
capturing, via a scope, a captured image including an imaging region where the imaging light reaches an imaging surface and a mask region where the imaging light does not reach the imaging surface;
determining an exposure state on the basis of information of pixels within a predetermined range of the captured image; and
executing processing by setting a type of the mask region as a first mask type determined on the basis of the captured image or a second mask type determined on the basis of an image acquired before the imaging according to the exposure state.

### REFERENCE SIGNS LIST

1 Medical image capturing system
4 Patient
10 Medical image capturing device
101 Scope
152 Imaging unit
161 Control unit
172 Light source
182 Evaluation region setting unit
184 Light receiving region setting unit
186 Focus region setting unit
192 Evaluation value generation unit
194 Light reception value generation unit
196 Image quality value generation unit
200-0 to 200-4 Evaluation frame
200 Captured image
201 Light amount control unit
202 Focus control unit
204 Gain control unit
206 Gradation control unit
210-0 to 210-8 Evaluation frame
220 Mask boundary
220-1 to 220-4 Mask boundary (edge)
230-1 to 230-4 Light amount detection region, focus detection region
240 Determination unit
242 Exposure state determination unit
244 Processing control unit
246 Type determination unit
511 Focus lens
ty1 to ty4 Mask type

1. A medical image capturing system that captures imaging light including a subject image obtained by irradiating a body cavity with illumination light from a light source unit, the medical image capturing system comprising:
an imaging unit that captures, via a scope, a captured image including an imaging region where the imaging light reaches an imaging surface and a mask region where the imaging light does not reach the imaging surface;
an exposure state determination unit that determines an exposure state on a basis of information of pixels within a predetermined range of the captured image; and
a processing unit that executes processing by setting a type of the mask region as a first mask type determined on a basis of the captured image or a second mask type determined on a basis of an image acquired before the imaging according to the exposure state.

2. The medical image capturing system according to claim 1, wherein the exposure state determination unit determines whether or not it is a state in which a type of the mask region can be correctly recognized.

3. The medical image capturing system according to claim 2, wherein in a case where the exposure state determination unit determines that the exposure state is a state in which the type of the mask region can be correctly recognized, the processing unit performs processing based on the first mask type.

4. The medical image capturing system according to claim 2, wherein in a case where the exposure state determination unit determines that the exposure state is a state in which a type of the mask region cannot be correctly recognized, the processing unit performs processing based on the second mask type.

5. The medical image capturing system according to claim 1, wherein the processing unit controls a light emission amount of the light source unit according to a detection region corresponding to a type of the mask region.

6. The medical image capturing system according to claim 5, wherein the processing unit reduces the light emission amount by PWM control.

7. The medical image capturing system according to claim 1, wherein
the imaging unit captures the captured image via a focus lens, and
the processing unit controls a focus of the focus lens according to a type of the mask region.

8. The medical image capturing system according to claim 7, wherein the processing unit stops focus control of the focus lens in a case where the exposure state determination unit determines that the exposure state is a state in which a type of the mask region cannot be correctly recognized.

9. The medical image capturing system according to claim 1, wherein the processing unit controls a gain of a signal output from the imaging unit according to a type of the mask region.

10. The medical image capturing system according to claim 1, wherein the processing unit executes gradation conversion of the captured image according to a type of the mask region.

11. The medical image capturing system according to claim 10, wherein in a case where the exposure state determination unit determines that the exposed state is a state in which a type of the mask region cannot be correctly recognized, the processing unit executes gradation conversion for widening a dynamic range more than that at a time of the determination.

12. The medical image capturing system according to claim 1, wherein the exposure state determination unit determines that the exposure state is a state in which a type of the mask region cannot be correctly recognized at least in a case where the first mask type and the second mask type are different and an evaluation value based on a pixel value in an evaluation frame in a central portion of the captured image is larger than a predetermined value.

13. The medical image capturing system according to claim 1, further comprising: a setting unit that sets a plurality of evaluation frames such that a mask boundary between the mask region of the first mask type and the imaging region is between the plurality of evaluation frames.

14. The medical image capturing system according to claim 13, wherein the exposure state determination unit sets, as a condition that the exposure state is a state in which a type of the mask region cannot be correctly recognized, that an evaluation value in the evaluation frame on the imaging region side of the two evaluation frames sandwiching the mask boundary of the first mask type is a predetermined value or more under control of the processing unit.

15. The medical image capturing system according to claim 14, wherein the exposure state determination unit sets, as a condition that the exposure state is a state in which a type of the mask region cannot be correctly recognized, that a pixel value average in an evaluation frame in a central portion of the captured image is a predetermined value or more.

16. The medical image capturing system according to claim 13, wherein the exposure state determination unit sets, as a condition that the exposure state is a state in which a type of the mask region can be correctly recognized, that an evaluation value in the evaluation frame on the mask region side of the two evaluation frames sandwiching the mask boundary of the first mask type is a predetermined value or less under control of the processing unit.

17. The medical image capturing system according to claim 13, wherein the setting unit sets the plurality of evaluation frames to be point-symmetric with respect to a corresponding point of an optical axis of the scope.

18. The medical image capturing system according to claim 1, further comprising: an estimation unit that estimates a type of the scope on a basis of a type of the mask region.

19. A method of capturing a medical image that captures imaging light including a subject image obtained by irradiating a body cavity with illumination light from a light source unit, the method comprising:
capturing, via a scope, a captured image including an imaging region where the imaging light reaches an imaging surface and a mask region where the imaging light does not reach the imaging surface;
determining an exposure state on a basis of information of pixels within a predetermined range of the captured image; and
executing processing by setting a type of the mask region as a first mask type determined on a basis of the captured image or a second mask type determined on a basis of an image acquired before the imaging according to the exposure state.
